(19) United States
(12) Patent Application Publication
(10) Pub. No.: US 2022/0212190 A1
(43) Pub. Date: Jul. 7, 2022

(54) MICROFLUIDIC CARTRIDGES FOR ENHANCED AMPLIFICATION OF POLYNUCLEOTIDE-CONTAINING SAMPLES

(71) Applicant: BECTON, DICKINSON AND COMPANY, Franklin Lakes, NJ (US)

(72) Inventors: Rohini Rao, Baltimore, MD (US); Joshua Keller, Pikesville, MD (US); Song Chong, Ellicott City, MD (US); Thomas Dawidczyk, Montclair, NJ (US); Karen L. Lenz, Cary, NC (US); Joel Daniel Krayer, New Milford, NJ (US); Edward Carrese, Monkton, MD (US)

(21) Appl. No.: 17/693,773

(22) Filed: Mar. 14, 2022

Related U.S. Application Data
(63) Continuation of application No. PCT/US2020/053399, filed on Sep. 30, 2020.
(60) Provisional application No. 62/909,628, filed on Oct. 2, 2019.

(51) Int. Cl. B01L 3/00

(52) U.S. Cl. CPC B01L 3/502715; B01L 3/502723; B01L 2300/087; B01L 7/52; B01L 2300/0819; B01L 2300/0887; B01L 2200/0684; B01L 2300/0883

(57) ABSTRACT

The technology described herein generally relates to microfluidic cartridges. The technology more particularly relates to a compressible pad applied to a microfluidic cartridge, wherein the microfluidic cartridge is configured to amplify nucleotides of interest, particularly from several biological samples in parallel, within microfluidic channels in the cartridge and permit detection of those nucleotides. Compressible pads of the present technology can be implemented in microfluidic cartridges having enhanced reaction chamber volumes, resulting in improved thermal uniformity and amplification efficiency in the cartridge. Assays using microfluidic cartridges of the present technology advantageously exhibit improved limit of detection (LOD) and improved limit of quantification (LOQ).

### CROSS REFERENCE TO RELATED APPLICATIONS

This application a continuation of International Application No. PCT/US2020/053399, filed Sep. 30, 2020, which claims the benefit of U.S. Provisional Application No. 62/909,628, filed Oct. 2, 2019, which are hereby incorporated by reference in their entirety.

### BACKGROUND

### Field

The technology described herein generally relates to microfluidic cartridges. In one aspect, the technology more particularly relates to a compressible pad applied to a microfluidic cartridge, wherein the microfluidic cartridge is configured to receive and amplify nucleotides of interest. In another aspect, the technology relates to a microfluidic cartridge having reaction chambers configured to receive and amplify larger volumes of fluid eluate from processed samples. Embodiments of the cartridges described herein can amplify nucleotides of interest from several biological samples in parallel, within microfluidic channels in the cartridge, and permit detection of those nucleotides.

### Description of the Related Art

The sensitivity of assays in molecular diagnostic tests is dependent on several factors. These factors include extraction efficiency during the processing of specimens to obtain amplification-ready samples, efficiency of amplification of the samples, and thermal uniformity achieved in a reaction volume during the amplification process, among other factors. Increasing the dimensions of the reaction volume contributes to improvements in the amplification efficiency, resulting in improved limit of detection (LOD) and improved limit of quantification (LOQ). Improving the uniformity and distribution of thermal communication between the reaction volume and a heat source contributes to improvements in thermal uniformity.

One current microfluidic cartridge implementation has reaction chambers having a reaction volume of about 4 μL. There are significant advantages associated with cartridges including reaction chambers with such small reaction volumes. As the volume of the reaction chamber decreases, however, challenges associated with achieving a desired analytical sensitivity can arise. At the same time, as the volume of the reaction chamber increases to achieve improved amplification efficiency and overcome target delivery limitations, challenges associated with achieving thermal uniformity can arise. There is a thus a need for microfluidic cartridges that overcome these challenges and achieve both improved amplification efficiency and thermal uniformity, resulting in assays having improved limit of detection (LOD) and improved limit of quantification (LOQ).

The discussion of the background to the technology herein is included to explain the context of the technology. This is not to be taken as an admission that any of the material referred to was published, known, or part of the common general knowledge as at the priority date of any of the claims.

Throughout the description and claims of the specification the word “comprise” and variations thereof, such as “comprising” and “comprises”, is not intended to exclude other additives, components, integers or steps.

### SUMMARY

The present technology includes methods and devices for improving pressure distribution across a microfluidic device, increasing thermal uniformity within the microfluidic device, and enhancing parameters of amplification performed in the microfluidic device. Implementations of the present technology improve features of microfluidic devices that amplify nucleotides of interest within microfluidic channels. The present technology includes methods and devices for improving detection of those nucleotides.

Microfluidic devices of the present technology can interact with a heating assembly that applies heat to a plurality of chambers in the microfluidic device where amplification occurs. The heating assembly can include an array of heaters configured to contact the microfluidic device. In some cases, the heating assembly is pressed against the microfluidic device to place the array of heaters in thermal communication with the microfluidic device. In other cases, the microfluidic device is pressed against the heating assembly to place the array of heaters in thermal communication with the microfluidic device. Embodiments of microfluidic devices according to the present technology can include a compressible pad that improves the distribution of pressure applied to the microfluidic device and increases the uniformity of heat delivered to the microfluidic device. Compressible pads of the present technology can increase the uniformity of pressure that is applied to the microfluidic device, resulting in reduced thermal losses and improving the consistency and efficiency of amplification occurring in the plurality of chambers of the microfluidic device.

Microfluidic devices of the present technology can also achieve improved assay sensitivity by increasing an amplification chamber volume from a volume of about 4 μL to a volume of about 25 μL, while still achieving optimal thermal uniformity across the chamber during an amplification process. The larger volume amplification chambers of the present technology can receive a larger volume of fluid eluate, containing DNA/RNA target analytes extracted from a specimen, thereby increasing assay sensitivity. In some cases, microfluidic devices of the present technology achieve a six-fold increase in reaction chamber volume as compared to current microfluidic devices. When these larger volume reaction chambers of the present technology are combined with improved pressure distribution and thermal uniformity associated with compressible pads of the present technology, assay performance increases as measured by improved limit of detection (LOD) and limit of quantification (LOQ).

Implementations of the improved microfluidic devices include a microfluidic cartridge. The microfluidic cartridge can include a first PCR reaction chamber. The microfluidic cartridge can include a second PCR reaction chamber. The microfluidic cartridge can include a first inlet, in fluid communication with the first PCR reaction chamber. The microfluidic cartridge can include a second inlet, in fluid communication with the second PCR reaction chamber. The microfluidic cartridge can include a compressible pad configured to increase compliance between the microfluidic cartridge and a heater.

In some embodiments, a microfluidic cartridge comprising a first side and an opposing, second side is provided. The microfluidic cartridge can include a first amplification chamber. The microfluidic cartridge can include a second amplification chamber. The microfluidic cartridge can include a first inlet disposed on the first side, in fluid communication with the first amplification chamber. The microfluidic cartridge can include a second inlet disposed on the first side, in fluid communication with the second amplification chamber. The microfluidic cartridge can include a compressible pad disposed on the first side. In some embodiments, the compressible pad is configured to provide more thorough and consistent heat transfer to the first amplification chamber and the second amplification chamber from a plurality of contact heat sources in contact with the second side of the microfluidic cartridge. In some embodiments, the compressible pad includes a first window above the first amplification chamber and a second window above the second amplification chamber. In some embodiments, the first window and the second window are configured to allow light to be transmitted through the first side of the microfluidic cartridge to and from the first amplification chamber and the second amplification chamber, respectively.

In some embodiments, the first amplification chamber and the second amplification chamber have a volume of about 25 μL. In some embodiments, the first amplification chamber and the second amplification chamber have a width dimension of about 3.5 mm, a depth dimension of about 0.83 mm, and a length dimension of about 10 mm. In some embodiments, the microfluidic cartridge comprises a label above the compressible pad. In some embodiments, the first amplification reaction chamber, the second amplification reaction chamber, the first inlet, and the second inlet are formed in a rigid substrate layer. In some embodiments, the second side of the microfluidic cartridge comprises a flexible laminate layer below the first amplification chamber and the second amplification chamber. In some embodiments, the compressible pad comprises a material with a Compression Force Deflection less than 30 psi. In some embodiments, the compressible pad comprises a material with a Compression Force Deflection less than 20 psi. In some embodiments, the compressible pad improves pressure distribution from a component of a diagnostic testing apparatus. In some embodiments, application of pressure to the compressible pad is configured to increase uniformity of the application of heat from the plurality of contact heat sources to the first amplification chamber and the second amplification chamber. In some embodiments, the compressible pad increases uniformity of the application of heat to the first amplification chamber and the second amplification chamber. In some embodiments, the compressible pad enhances PCR amplification which relies on rapid temperature cycling.

In some embodiments, a method for amplifying on a plurality of polynucleotide-containing samples is provided. The method can comprise introducing the plurality of samples into a microfluidic cartridge, wherein the cartridge comprises a plurality of amplification chambers configured to permit thermal cycling of the plurality of samples independently of one another. The method can comprise moving the plurality of samples into the respective plurality of amplification chambers. The method can comprise amplifying polynucleotides contained with the plurality of samples, by application of successive heating and cooling cycles to the amplification chambers. The method can comprise compressing a pad of the microfluidic cartridge during amplification. In some embodiments, the method can comprise applying pressure to the compressible pad to increase contact between the microfluidic cartridge and a substrate comprising one or more heaters. In some embodiments, the method can comprise applying pressure to the compressible pad to increase thermal uniformity. In some embodiments, the method can comprise applying pressure to the compressible pad to enhance amplification of the plurality of polynucleotide-containing samples.

In some embodiments, a system is provided. The system can include a microfluidic substrate. The microfluidic substrate can include a first PCR reaction chamber. The microfluidic substrate can include a second PCR reaction chamber. The microfluidic substrate can include a first inlet, in fluid communication with the first PCR reaction chamber. The microfluidic substrate can include a second inlet, in fluid communication with the second PCR reaction chamber. The microfluidic substrate can include a compressible pad. In some embodiments, the microfluidic cartridge is configured for use with an apparatus. The apparatus can include a bay configured to receive the microfluidic cartridge. The apparatus can include at least one heat source thermally coupled to the cartridge and configured to apply heat cycles that carry out PCR on one or more polynucleotide-containing sample in the cartridge. The apparatus can include a detector configured to detect presence of one or more polynucleotides in the one or more samples. The apparatus can include a processor coupled to the heat source and configured to control heating of one or more regions of the microfluidic cartridge.

In some embodiments, the compressible pad is configured to improve contact between the bay and the microfluidic cartridge. In some embodiments, the compressible pad is configured to improve contact between the at least one heat source and the cartridge. In some embodiments, the compressible pad is configured to be compressed by the detector which is disposed above the cartridge during detection. In some embodiments, the detector is configured to move down and make physical contact with the cartridge to compress the compressible pad. In some embodiments, the cartridge is configured to move up and make physical contact with the detector to compress the compressible pad. In some embodiments, the compressible pad is configured to be compressed by another component of the apparatus which applies pressure to the cartridge.

The details of one or more embodiments of the technology are set forth in the accompanying drawings and further description herein. Other features, objects, and advantages of the technology will be apparent from the description and drawings, and from the claims.

### BRIEF DESCRIPTION OF THE DRAWINGS

FIG. 1A shows a plan view of an example multi-lane microfluidic cartridge;

FIG. 1B shows a close-up view of a portion of the cartridge of FIG. 1A illustrating reaction chambers;

FIG. 2A shows a plan view of another example of a multi-lane cartridge with reaction chambers having enhanced features;

FIG. 2B shows a close-up view of a portion of the cartridge of FIG. 2A illustrating reaction chambers;

FIG. 2C shows an example reaction chamber of the cartridge of FIG. 2A.

FIG. 2D shows a view of still another example of a multi-lane microfluidic cartridge with reaction chambers of varying volumes.

FIG. 3A shows a cut-away layer construction view of a further example of a cartridge including a compressible pad;

FIG. 3B is an exploded view of the cartridge of FIG. 3A;

FIG. 4 shows the compressible pad of the cartridge of FIG. 3A;

FIG. 5A shows an example heater module of a receiving bay;

FIGS. 5B-5D show an example system with two receiving bays;

FIG. 6 shows an optical detector;

FIGS. 7A-7C show results for assay testing for an analyte of interest without a compressible pad;

FIGS. 8A-8D show results for assay testing for the analyte of interest with a low durometer silicone compressible pad;

FIGS. 9A-9D show results for assay testing for the analyte of interest with a PORON® foam compressible pad.

FIGS. 10-37 show views of still a further example of a multi-lane cartridge with reaction chambers having enhanced features.

FIGS. 38A-38B show aspects of an example heater array and heater element fine structure of a heating apparatus configured to apply heat to a microfluidic cartridge.

### DETAILED DESCRIPTION

The present technology relates to a microfluidic device that is configured to carry out amplification, such as by PCR, of one or more polynucleotides from one or more samples. Unless specifically made clear to the contrary, where the term PCR is used herein, any variant of PCR including but not limited to real-time and quantitative, and any other form of polynucleotide amplification is intended to be encompassed.

The microfluidic cartridge can be configured so that it receives thermal energy from one or more heating elements present in an external apparatus with which the cartridge is in thermal communication. An exemplary such apparatus is further described herein; additional embodiments of such an apparatus are described in U.S. patent application Ser. No. 11/985,577, entitled “Microfluidic System for Amplifying and Detecting Polynucleotides in Parallel” and filed on Nov. 14, 2007, the specification of which is incorporated herein by reference. The present technology provides for an apparatus for detecting polynucleotides in samples, particularly from biological samples. The technology more particularly relates to microfluidic systems that carry out PCR on nucleotides of interest within microfluidic channels and detect those nucleotides. The apparatus includes a microfluidic cartridge that is configured to accept a plurality of samples, and which can carry out PCR on each sample individually, or a group of, or all of the plurality of samples simultaneously. U.S. patent application Ser. No. 11/940,315, entitled “Heater Unit for Microfluidic Diagnostic System” and filed on Nov. 14, 2007, is incorporated herein by reference. U.S. patent application Ser. No. 11/940,310, entitled “Microfluidic Cartridge and Method of Using Same” and filed on Nov. 14, 2007, is incorporated herein by reference. The present technology provides for a microfluidic substrate configured to carry out PCR on a number of polynucleotide-containing samples in parallel. The substrate can be a single-layer substrate in a microfluidic cartridge. Also provided are a method of making a microfluidic cartridge including such a substrate. U.S. patent application Ser. No. 11/728,964, entitled “Integrated System for Processing Microfluidic Samples and Methods of Using Same” and filed on Mar. 26, 2007, is incorporated herein by reference. The present technology provides an integrated apparatus for processing polynucleotide-containing samples, and for providing a diagnostic result thereon.

By cartridge is meant a unit that may be disposable, or reusable in whole or in part, and that is configured to be used in conjunction with some other apparatus that has been suitably and complementarily configured to receive and operate on (such as deliver energy to) the cartridge.

By microfluidic, as used herein, is meant that volumes of sample, and/or reagent, and/or amplified polynucleotide are from about 0.1 μl to about 999 μl, such as from 1-100 μl, or from 1-50 μl. In some embodiments, the volume is between 0 and 10 μl for smaller wells and between 10 and 30 μl for wider, deeper wells as described herein. Similarly, as applied to a cartridge, the term microfluidic means that various components and channels of the cartridge, as further described herein, are configured to accept, and/or retain, and/or facilitate passage of microfluidic volumes of sample, reagent, or amplified polynucleotide. Certain embodiments herein can also function with nanoliter volumes (in the range of 10-500 nanoliters, such as 100 nanoliters).

One aspect of the present technology relates to a microfluidic cartridge having two or more sample lanes arranged so that analyses can be carried out in two or more of the lanes in parallel, for example simultaneously, and wherein each lane is independently associated with a given sample (hereinafter referred to as a “sample lane”).

A sample lane is an independently controllable set of elements by which a sample can be analyzed, according to methods described herein as well as others known in the art. A sample lane includes at least a sample inlet, and a microfluidic network having one or more microfluidic components, as further described herein.

The cartridge can include a plurality of microfluidic networks, each network having various components, and each network configured to carry out PCR on a sample in which the presence or absence of one or more polynucleotides is to be determined.

Embodiments of the present technology include a cartridge having a plurality of sample lanes, hereinafter referred to as a “multi-lane cartridge.” It will be understood, however, that embodiments of the present technology can be implemented in a cartridge including no more than one sample lane. A multi-lane cartridge is configured to accept a number of samples in series or in parallel, simultaneously or consecutively. In some embodiments the multi-lane cartridge is configured to accept 24 samples, or any other suitable number of samples. In some instances, the multi-lane cartridge is configured to accept at least a first sample and a second sample, where the first sample and the second sample each contain one or more polynucleotides in a form suitable for amplification. The polynucleotides in question may be the same as, or different from one another, in different samples and hence in different sample lanes of the cartridge. The cartridge can process each sample by increasing the concentration of a polynucleotide to be determined and/or by reducing the concentration of inhibitors relative to the concentration of polynucleotide to be determined.

The multi-lane cartridge includes at least a first sample lane having a first microfluidic network and a second sample lane having a second microfluidic network, each of the first microfluidic network and the second microfluidic network including features described herein, and wherein the first microfluidic network is configured to amplify polynucleotides in the first sample, and wherein the second microfluidic network is configured to amplify polynucleotides in the second sample.

In various embodiments, the microfluidic network can be configured to couple heat from an external heat source to a sample mixture comprising PCR reagents and a neutralized polynucleotide sample under thermal cycling conditions suitable for creating PCR amplicons from the neutralized polynucleotide sample.

At least the external heat source may operate under control of a computer processor, configured to execute computer readable instructions for operating one or more components of each sample lane, independently of one another, and for receiving signals from a detector that measures fluorescence from one or more of the PCR reaction chambers.

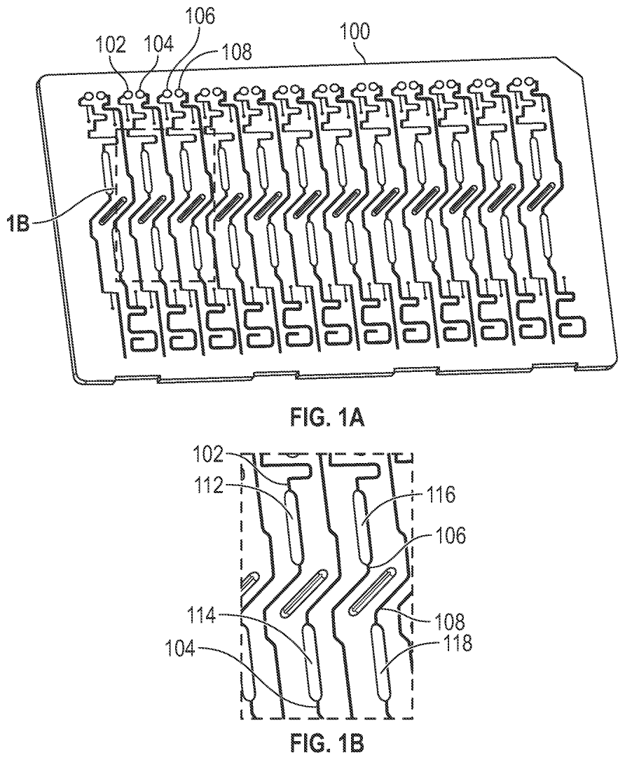

A non-limiting implementation of a microfluidic cartridge according to the present technology will now be described with reference to FIGS. 1A and 1B. FIG. 1A shows a plan view of a microfluidic cartridge 100 including twenty-four independent sample lanes, including sample lanes 102, 104, 106, 108. FIG. 1B shows a close-up view of a portion of the cartridge 100 of FIG. 1A illustrating reaction chambers 112, 114, 116, 118 of adjacent sample lanes 102, 104, 106, 108. The microfluidic network in each sample lane is typically configured to carry out amplification, such as by PCR, on a PCR-ready sample. The microfluidic network in each sample lane can accept and amplify a nucleic acid-containing sample extracted from a specimen using any suitable method. In examples of cartridges that accept a PCR-ready sample, the sample can include a mixture including PCR reagents and the neutralized polynucleotide sample, suitable for subjecting to thermal cycling conditions that create PCR amplicons from the neutralized polynucleotide sample. In one example, the PCR-ready sample includes a PCR reagent mixture comprising a polymerase enzyme, a positive control plasmid, a fluorogenic hybridization probe selective for at least a portion of the plasmid and a plurality of nucleotides, and at least one probe that is selective for a polynucleotide sequence. Exemplary probes are further described herein. In embodiments of the present technology, the microfluidic network is configured to couple heat from an external heat source with the mixture comprising the PCR reagent and the neutralized polynucleotide sample under thermal cycling conditions suitable for creating PCR amplicons from the neutralized polynucleotide sample.

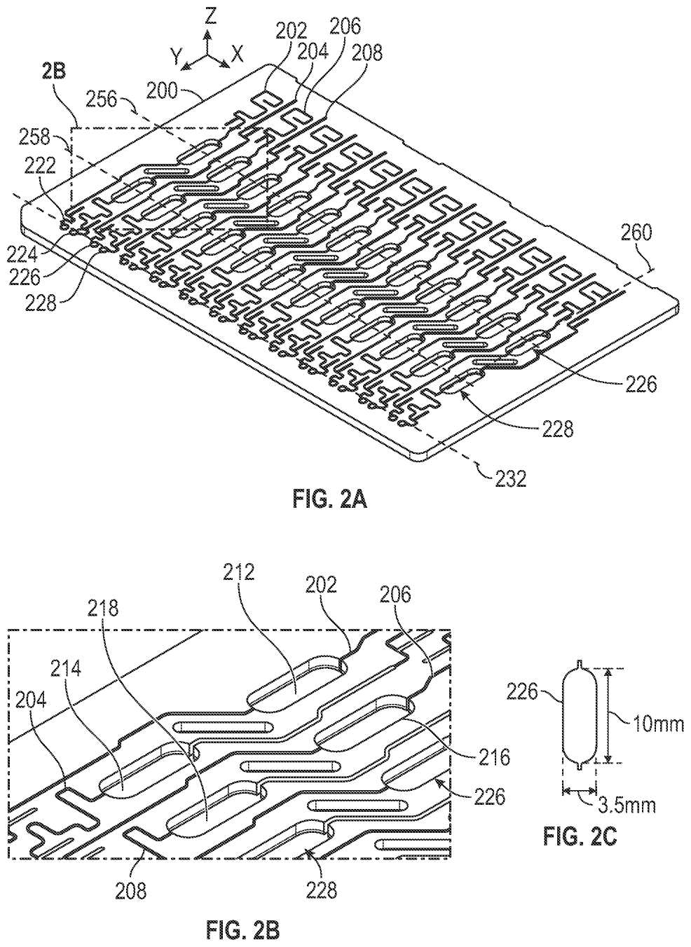

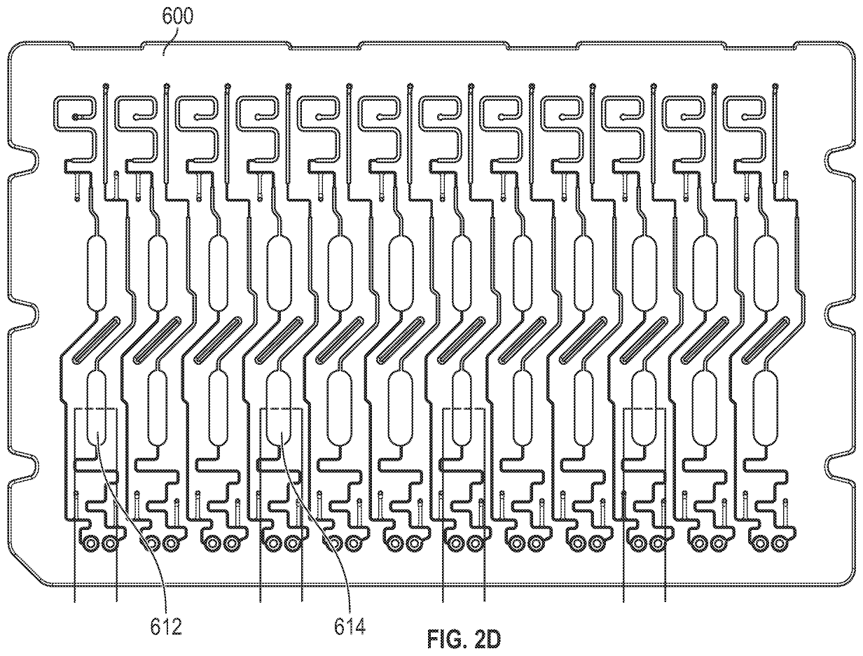

Another non-limiting implementation of a microfluidic cartridge according to the present technology will now be described with reference to FIGS. 2A and 2B. FIG. 2A shows a plan view of a microfluidic cartridge 200 containing twenty-four independent sample lanes, including sample lanes 202, 204, 206, 208. FIG. 2B shows a close-up view of a portion of the cartridge 200 of FIG. 2A illustrating reaction chambers 212, 214, 216, 218 of adjacent sample lanes 202, 204, 206, 208. The sample lanes of the cartridge 200 each include a dedicated sample inlet configured to accept a sample. For example, the sample lanes 202, 204, 206, and 208 include sample inlets 222, 224, 226, 228, respectively, where each sample inlet is configured to independently accept a sample. The cartridge 200 may be referred to as a multi-lane PCR cartridge with dedicated sample inlets. The sample inlets can be configured to accept a liquid transfer member (not shown) such as a syringe, a pipette, or a PCR tube containing a PCR ready sample. In embodiments of cartridges according to the present technology, one inlet operates in conjunction with a single sample lane.

In the embodiment of FIG. 2A, each reaction chamber 212, 214, 216, 218 has at least one dimension which is greater than each reaction chamber 112, 114, 116, 118 of the embodiment of FIG. 1A. The reaction chambers 212, 214, 216, 218 can be considered wider, wherein the width dimension is measured along an x-axis of the microfluidic cartridge. The reaction chambers 212, 214, 216, 218 can be considered deeper, wherein the depth dimension is measured along a z-axis of the microfluidic cartridge. In some embodiments, the reaction chambers 212, 214, 216, 218 can be considered longer, wherein the length dimension is measured along a y-axis axis of the microfluidic cartridge. The length and width dimensions can be disposed along perpendicular axes. In the illustrative embodiment, the reaction chambers 212, 214, 216, 218 are wider and deeper than the reaction chambers 112, 114, 116, 118. Each reaction chamber 212, 214, 216, 218 can have a greater volume than each reaction chamber 112, 114, 116, 118. As a result, each reaction chamber 212, 214, 216, 218 can hold a greater volume of fluid than each reaction chamber 112, 114, 116, 118.

In some embodiments, the cartridge 200 includes an increased thickness to accommodate the deeper reaction chambers of FIG. 2A, where the thickness dimension is measured along the z-axis of the microfluidic cartridge. The cartridge 200 can have thickness of about 1.68 mm thick compared to cartridge 100 which can have a thickness of about 1.24 mm. In some embodiments, the thicker cartridge can have poorer thermal performance characteristics than the thinner cartridge, including edge effect failures (outside sample lanes), reverse edge effect failures (inside sample lanes), and random failures. Embodiments of a compressible pad according to the present technology, as described herein, can improve thermal conductivity and/or thermal coupling between the cartridge 200 and a heating apparatus to reduce these failures.

The reaction chambers 212, 214, 216, 218 can have any shape. In the illustrative embodiment, the reaction chambers 212, 214, 216, 218 can have an oblong shape. The edges of the reaction chambers 212, 214, 216, 218 can be rounded. Other shapes of reaction chambers are contemplated.

The chambers 212, 214, 216, 218 in adjacent sample lanes 202, 204, 206, 208 are staggered with respect to one another. In some embodiments, the sample inlets are all disposed along a single line 232 parallel to the x-axis of the microfluidic cartridge. The 24-lane cartridge has two banks 226, 228 of twelve PCR reaction chambers, shown in FIGS. 2A and 2B. Each network can include a reaction chamber. In some embodiments, each network can include two valves on either side of the reaction chamber. Valves are normally open initially and close the channel upon actuation. The valves can include microvalves. In some embodiments, each network can include an outlet or vent. In some examples, the outlet or vent can allow gas in the microfluidic network to escape the microfluidic network as sample is moved through the microfluidic network from an inlet to a chamber. In some examples, the outlet or vent can allow an amplified sample to be removed from the microfluidic network.

In some embodiments, the reaction chamber 212 in the first bank of reaction chambers 226 is aligned with the reaction chamber 214 in the second bank of PCR sample lanes. The reaction chambers 212, 214 can be aligned transverse to the single line 232 of sample inlets. Adjacent networks can form staggered reaction chambers as shown in the illustrated embodiments. In some embodiments, the 24-lane cartridge has two banks of twelve reaction chambers 226, 228. One first bank of twelve reaction chambers 226, 228 is closer to the inlets. The other bank of twelve reaction chambers 226, 228 is farther from the inlets. The first bank of twelve reaction chambers 226 can be axially aligned along a first axis 256 and the second bank of twelve reaction chambers 228 can be axially aligned along a second axis 258. The reaction chamber 212 of the first bank of twelve reaction chambers 226 and the reaction chamber 214 of the second bank of reaction chambers 228 can be aligned along a third axis 260. The third axis can be transverse or perpendicular to the first axis and/or the second axis. Other configurations are contemplated.

As one example, the reaction chambers 112, 114, 116, 118 can each be a 4 microliter PCR reaction chamber. As one example, the reaction chambers 112, 114, 116, 118 can each be about 1.5 mm wide, about 0.30 mm (300 microns) deep, and approximately 10 mm long. The volume of the reaction chambers can be approximately 4 μl. It would be understood that these dimensions and layout are exemplary, and deviations from those shown are consistent with an equivalent manner of operation of such a cartridge. The microfluidic cartridge 100 can permit PCR to be carried out in a concentrated reaction volume (˜4 μl) and enable rapid thermocycling, at ˜20 seconds per cycle. As another example, typical dimensions of a reaction chamber are 150 μ deep by 700 μ wide, and a typical volume is ˜1.6 μl. Channels of a microfluidic network in a sample lane of cartridge 100 can have at least one sub-millimeter cross-sectional dimension. For example, channels of such a network may have a width and/or a depth of less than 1 mm (e.g., about 750 microns or less, about 500 microns, or less, or about 250 microns or less).

In implementations of the present technology, the reaction chambers 212, 214, 216, 218 can have an increased width and/or an increased depth (but the same or similar length) relative to the reaction chambers 112, 114, 116, 118 of microfluidic cartridge 100. In a first example, the reaction chambers 212, 214, 216, 218 are each approximately 3.5 mm wide, approximately 0.54 mm (540 microns) deep, and approximately 10 mm long. The volume of the reaction chamber is approximately 16.8 μL. In a second example, the reaction chambers 212, 214, 216, 218 are each approximately 2.5 mm wide, approximately 0.86 mm (860 microns) deep, and approximately 10 mm long. The volume of the reaction chamber is approximately 18.6 μL. In some embodiments, the reaction chambers 212, 214, 216, 218 can each be a PCR reaction chamber having a volume of about 25 microliters. In a third example illustrated in FIG. 2C, the reaction chambers 212, 214, 216, 218 are each approximately 3.5 mm wide, approximately 0.83 mm (830 microns) deep, and approximately 10 mm long. The volume of the reaction chamber is approximately 25.2 μL. In a fourth example, the reaction chambers 212, 214, 216, 218 are each approximately 2.5 mm wide, approximately 1.35 mm (1350 microns) deep, and approximately 10 mm long. The volume of the reaction chamber is approximately 25.2 μL. In the context of viral load assay testing described in non-limiting examples below, it was determined that the third example exhibited optimal performance characteristics for improved viral load assay testing.

The above-described example reaction chambers are summarized in the following table.

TABLE 1

| | Volume (μL) | Width (mm) | Depth (mm) | Length (mm) |
|---|---|---|---|---|
| Cartridge 100 | 4.2 | 1.5 | 0.3 | 10.00 |
| Cartridge 200 | 16.8 | 3.5 | 0.54 | 10.00 |
| Example 1 | | | | |
| Cartridge 200 | 18.6 | 2.5 | 0.86 | 10.00 |
| Example 2 | | | | |
| Cartridge 200 | 25.2 | 3.5 | 0.83 | 10.00 |
| Example 3 | | | | |
| Cartridge 200 | 25.2 | 2.5 | 1.35 | 10.00 |
| Example 4 | | | | |

Embodiments of microfluidic cartridges described herein can include reaction chambers that have different volumes. For example, in one non-limiting embodiment illustrated in FIG. 2D, a microfluidic cartridge 600 includes reaction chambers 612 having a volume of approximately 4 μL and reaction chambers 614 having a volume of approximately 16 μL. It will be understood that embodiments of the microfluidic cartridge 600 are not limited to the particular arrangement of reaction chambers illustrated in FIG. 2D, and other arrangements and combinations of reaction chamber volumes are possible.

In some embodiments, the width of the reaction chambers 212, 214, 216, 218 can be between 1 and 4 mm (e.g., 1 mm, 1.5 mm, 2 mm, 2.5 mm, 3 mm, 3.5 mm, 4 mm, between 1 and 2 mm, between 2 and 3 mm, between 3 and 4 mm, or any range of two of the foregoing values.) In some embodiments, the depth of the reaction chambers 212, 214, 216, 218 can be between 0 and 2 mm (e.g., 0.1 mm, 0.2 mm, 0.3 mm, 0.4 mm, 0.5 mm 0.6 mm, 0.7 mm, 0.8 mm, 0.9 mm, 1.0 mm, 1.1 mm, 1.2 mm, 1.3 mm, 1.4 mm, 1.5 mm 1.6 mm, 1.7 mm, 1.8 mm, 1.9 mm, 2 mm, 0.25 mm, 0.5 mm, 0.75 mm, 1 mm, 1.25 mm, 1.5 mm, 1.75 mm, between 0 and 0.5 mm, between 0.5 and 1 mm, between 1 and 1.5 mm, or any range of two of the foregoing values.) In some embodiments, the length of the reaction chambers 212, 214, 216, 218 can be between 8 mm and 12 mm (e.g., 8 mm, 9 mm, 10 mm, 11 mm, 12 mm, between 9 and 11 mm, approximately 10 mm or any range of two of the foregoing values.) In some embodiments, the volume of the reaction chambers 212, 214, 216, 218 can be between 10 μl and 30 μl (e.g., 10 μl, 11 μl, 12 μl, 13 μl, 14 μl, 15 μl, 16 μl, 17 μl, 18 μl, 19 μl, 20 μl, 21 μl, 22 μl, 23 μl, 24 μl, 25 μl, 26 μl, 27 μl, 28 μl, 29 μl, 30 μl, between 10 μl and 15 μl, between 15 μl, and 20 μl, between 20 μl and 25 μl, between 25 μl and 30 μl, or any range of two of the foregoing values.) It would be understood that these dimensions and layouts are exemplary, and deviations from those shown are consistent with an equivalent manner of operation of such a cartridge. In some embodiments, each reaction chamber 112, 114, 116, 118 has a volume of 4 ml. In some embodiments, each reaction chamber 212, 214, 216, 218 has a volume of 25 ml, or approximately six times greater than the reaction chambers 112, 114, 116, 118.

### Enhanced Microfluidic Cartridges Having Larger Volume Reaction Chambers

The microfluidic cartridge 200 can be designed for nucleic acid amplification. As described herein, the microfluidic cartridge 200 has an increased volume PCR reaction chamber of approximately 25.2 μl total volume, allowing a larger volume of fluid eluate to be amplified from a specimen in process. In particular, embodiments of microfluidic cartridge 200 can ensure that a greater percentage of liquid eluate from a sample processing procedure can be loaded into, and amplified within, the PCR reaction chamber. In some cases, there is a six-fold increase in the volume of liquid eluate that can be amplified. Implementations of enhanced microfluidic cartridges of the present technology have larger volume reaction chambers and can therefore accommodate larger liquid eluate input. As a result, enhanced microfluidic cartridges of the present technology allow for a more consistent amplification process across samples and cartridges, reduce variation in the amplification process across samples and cartridges, and improve performance of assays overall.

In implementations where the microfluidic cartridge 200 includes a plastic substrate layer, the geometry of each reaction chamber 212, 214, 216, 218 is formed within the plastic substrate layer on all but one side where each reaction chamber 212, 214, 216, 218 is sealed by a laminate layer, as described herein. Sample nucleic acid and PCR reagent mix can be loaded into the chamber through inlet ports and microfluidic channels. Each reaction chamber 212, 214, 216, 218 can be sealed by heat activated wax valves that spread into the fluid path and cool on either side of the chamber. As described herein, heat is applied to each reaction chamber 212, 214, 216, 218 through the laminate layer on a bottom side of the cartridge 200 to perform the PCR reaction, and fluorescence change is measured via external optics disposed over the chamber on a top side of the cartridge 200.

The microfluidic cartridge 100 accommodates approximately 4.2 μl of reaction volume per reaction chamber 112, 114, 116, 118. In some embodiments, the microfluidic cartridge 200 achieves improved analytical sensitivity relative to the microfluidic cartridge 100. In some embodiments, the larger PCR chamber capacity of the microfluidic cartridge 200 overcomes target delivery limitations of the microfluidic cartridge 100. In some embodiments, the microfluidic cartridge 200 achieves improved performance of sensitivity by increasing the PCR chamber volume to approximately 25.2 μl. In some embodiments, a larger volume PCR chamber is desired as more DNA/RNA input from the specimen extraction can increase sensitivity. In some embodiments, a larger volume PCR chamber provides better performance. In some embodiments, a larger volume PCR chamber improves a limit of detection for an amplification performed in the larger volume PCR chamber. In some embodiments, a larger volume PCR chamber improves a limit of quantification for an amplification performed in the larger volume PCR chamber. In some embodiments, a larger volume PCR chamber improves PCR efficiency.

The sensitivity of assays is dependent on several contributing factors, including extraction efficiency, PCR efficiency, and thermal uniformity. In some embodiments, increased dimension(s) of the chamber is one contributor to improve PCR efficiency resulting in improved limit of detection and limit of quantification. In some embodiments, the microfluidic cartridge 200 achieves a six-time volume increase compared to the microfluidic cartridge 100. Other configurations are contemplated (e.g., two-fold volume increase, three-fold volume increase, four-fold volume increase, five-fold volume increase, six-fold volume increase, seven-fold volume increase, eight-fold volume increase, or any range of two or more foregoing values). There is an upper limit to the amount the dimension of the chamber can be increased while still achieving optimal thermal uniformity throughout the chamber during each cycle of an amplification protocol. This is particularly true in the case of amplification protocols with particular, optimized cycle times to achieve reliable PCR. In some embodiments, the microfluidic cartridge 200 can accommodate larger eluate input from a sample processing procedure performed on a specimen. In some embodiments, the microfluidic cartridge 200 can improve limit of detection and limit of quantification of assays. In some embodiments, the microfluidic cartridge 200 can ensure that a greater percentage of the liquid eluate from sample processing can be loaded into the increased dimension chamber. In some embodiments, the microfluidic cartridge 200 can facilitate more consistent PCR amplification. In some embodiments, the microfluidic cartridge 200 can reduce variation in PCR amplification. In some embodiments, the microfluidic cartridge 200 can improve overall performance of the assay performed on a sample.

The reaction chamber in a given sample lane has length, width, and depth dimensions to permit PCR to amplify polynucleotides present in a sample received in the reaction chamber. The upper portion of each reaction chamber includes a window that permits detection of fluorescence from a fluorescent substance in the reaction chamber when a detector is situated above the window. It is to be understood that other configurations of windows are possible including, but not limited to, a single window that straddles each PCR reactor across the width of cartridge.

The sample inlets of adjacent sample lanes are spaced apart from one another to prevent any contamination of one sample inlet during introduction of a sample into an adjacent sample inlet in the cartridge. In some embodiments, the sample inlets are configured so as to prevent subsequent inadvertent introduction of sample into a given sample lane after a sample has already been introduced into that sample lane. In some embodiments, the multi-sample cartridge is designed so that the spacing between the centroids of sample inlets is 6 mm, which is an industry-recognized standard. This means that, in certain embodiments the center-to-center distance between inlet holes in the cartridge is 6 mm. The inlet holes can be manufactured conical in shape with an appropriate conical angle so that industry-standard pipette tips (2 μl, 20 μl, 200 μl, volumes, etc.) fit snugly therein. The cartridge herein can be adapted to suit other, later-arising, industry standards not otherwise described herein, as would be understood by one of ordinary skill in the art.

In some embodiments, the microfluidic cartridge includes a first, second, and third layer that together define a plurality of microfluidic networks, each network having various components configured to carry out PCR on a sample having one or more polynucleotides whose presence is to be determined. As described herein, the microfluidic cartridge can include a fourth layer designed to improve pressure distribution, increase thermal uniformity, and enhance PCR amplification. In some embodiments, the fourth layer is a compressible pad. While four layers are described, the microfluidic cartridge can include fewer layers and one or more layers can be combined in a single integrated layer. While four layers are described, additional layers can be included and one or more layers can be separated into two or more layers.

The cartridge includes one or more sample lanes, wherein each sample lane is independently associated with a given sample for simultaneous processing, and each sample lane contains an independently configured microfluidic network. The cartridge typically processes the one or more samples by increasing the concentration of (such as by amplification) one or more polynucleotides to be determined, as present in each of the samples.

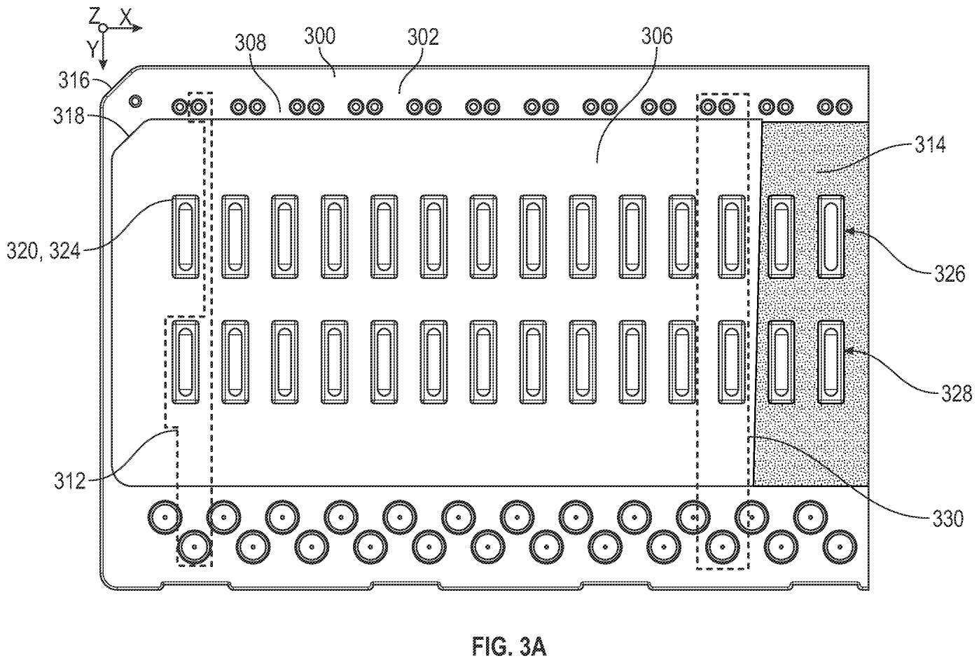

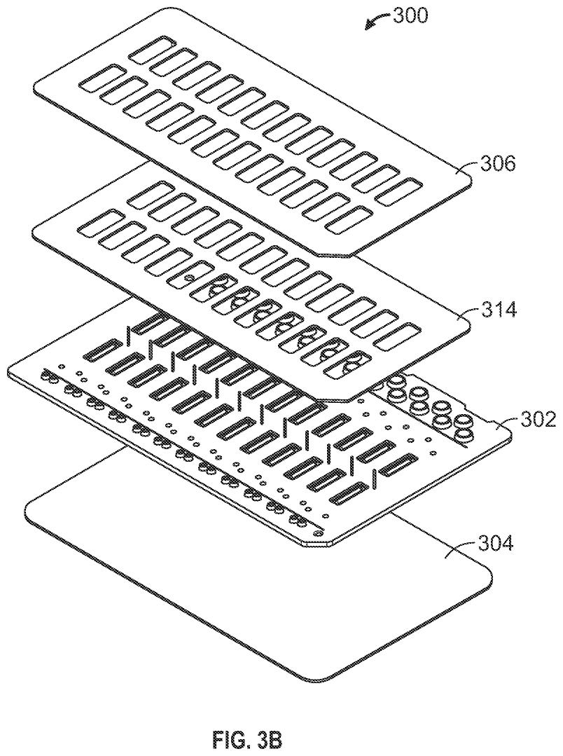

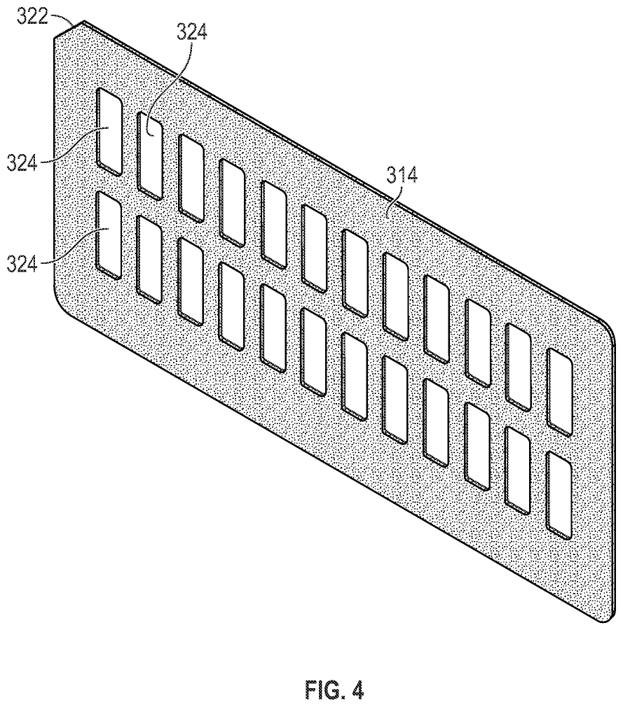

The cartridge herein includes embodiments having three or more layers in their construction, as shown in the embodiment 300 of FIGS. 3A and 3B. The cartridge 300 includes a substrate 302, a laminate 304 (not visible in FIG. 3A), and a label 306. In cartridge 300, a microfluidic substrate 302 has an upper side 308 and, on an opposite side of the substrate, a lower side 310 (not visible in FIG. 3A). The substrate 302 includes a plurality of microfluidic networks, arranged into a corresponding plurality of sample lanes 312. The cartridge 300 includes a plurality of cartridge lanes 330. In this non-limiting embodiment, the cartridge 300 includes 12 cartridge lanes 330. In this non-limiting embodiment, each cartridge lane 330 corresponds to a region of the cartridge 300 that includes 2 sample lanes 312. In this non-limiting embodiment, the cartridge 300 includes 24 sample lanes 312 arranged into 12 parallel cartridge lanes 330. The cartridge 300 can include a laminate 304 attached to the lower side 310 of the substrate 302 to seal various components (for example, valves) of the microfluidic networks. The laminate 304 can provide an effective thermal transfer layer between a dedicated heating element (further described herein) and components in the microfluidic networks. The cartridge 300 can include a label 306, attached to the upper side 308 of the substrate 302. In some embodiments, each reaction chamber is formed within the microfluidic substrate layer on all but one or more sides where each reaction chamber is sealed off by one or more additional layers. In some embodiments, each reaction chamber is sealed by the laminate 304. In some embodiments, each reaction chamber is sealed by the laminate 304. In some embodiments, each reaction chamber is sealed by the label 306.

The cartridge 300 can include a compressible pad 314. In some embodiments, the compressible pad 314 is placed above the upper side 308 of the substrate 302. In some embodiments, the compressible pad 314 is placed between the upper side 308 of the substrate 302 and the label 306. In one example embodiment, the compressible pad 314 is placed below the label 306. In another example embodiment, the compressible pad 314 is placed above the label 306. In embodiments where the compressible pad 314 is placed above the label 306, the label 306 can cover and seal holes that are used in the manufacturing process to load components such as valves of the microfluidic networks with thermally responsive materials. In such embodiments where the compressible pad 314 is placed above the label 306, markings (described in detail below) that would ordinarily be included on the label 306 can be included on the compressible pad 314. In some embodiments, each reaction chamber is sealed by the compressible pad 314 when the compressible pad 314 is disposed under the label 306.

In some embodiments, not shown, the compressible pad 314 is placed below the lower side 310 of the substrate 302. In some embodiments, the compressible pad 314 is placed between the lower side 310 of the substrate 302 and the laminate 304. In some embodiments, the compressible pad 314 is above the laminate 304. In some embodiments, the compressible pad 314 is below the laminate 304. In such embodiments, the compressible pad can be formed of a thermally conductive material or include thermally conductive properties.

Thus, embodiments of microfluidic cartridges herein include embodiments consisting of layers including a substrate 302, a laminate 304, and a label 306, wherein the compressible pad 314 is placed adjacent to at least one of the layers. In some embodiments, the microfluidic cartridge consists essentially of four layers: a substrate, a laminate, a label, and a compressible pad. In some embodiments, the microfluidic cartridge comprises four layers: a substrate, a laminate, a label, and a compressible pad.

The microfluidic substrate layer 302 is typically injection molded out of a plastic, preferably a zeonor plastic (cyclic olefin polymer), and contains a number of microfluidic networks (shown in FIGS. 1A and 2A). In some embodiments, as described herein, the substrate 302 comprises twenty-four reaction chambers that contain material for PCR amplification. Each microfluidic network includes a reaction chamber and associated channels. In some embodiments, the microfluidic networks include one or more valves. The valves, when present, can be disposed on a first (e.g., lower) side (disposed towards the laminate). In some embodiments, the microfluidic networks include loading holes for loading wax or other thermally responsive substances in the valve. In some embodiments, the microfluidic networks include one or more vent channels. In some embodiments, the microfluidic networks include one or more liquid inlet holes, on a second (e.g., upper) side (disposed toward the label layer). Typically, in a given cartridge, all of the microfluidic networks together, including the reaction chambers and the inlet holes, are defined in a single substrate layer, substrate 302.

The substrate 302 can be formed of a material that enhances rigidity of the substrate (and hence the cartridge). The material from which the substrate 302 is formed can be rigid or non-deformable. Rigidity is advantageous because it facilitates effective and uniform contact with a heating assembly as further described herein. In some embodiments, the substrate 302 is impervious to air or liquid, so that entry or exit of air or liquid during operation of the cartridge is only possible through the inlets or the various vents. The material from which the substrate 302 is formed can be non-venting to air and other gases. Use of a non-venting material is also advantageous because it reduces the likelihood that the concentration of various species in liquid form will change during analysis. In some embodiments, the substrate 302 has a low autofluorescence to facilitate detection of polynucleotides during an amplification reaction performed in the microfluidic circuitry defined therein. Use of a material having low auto-fluorescence is also important so that background fluorescence does not detract from measurement of fluorescence from the analyte of interest.

The substrate 302 can have an area of reduced thickness to facilitate detection. In some embodiments, the area of reduced thickness can be above each reaction chamber in each sample lane. In some embodiments, the area of reduced thickness can have an oblong or elongate shape. The area of reduced thickness can have a surface area equal or greater than the area of the corresponding reaction chamber.

The laminate layer 304 can be a heat sealable laminate layer. The laminate layer 304 can be typically between about 100 and about 125 microns thick. The laminate layer 304 can be attached to the bottom surface of the microfluidic substrate 302 using, for example, heat bonding, pressure bonding, or a combination thereof. The laminate layer 304 may also be made from a material that has an adhesive coating on one side only, that side being the side that contacts the underside of the substrate 302. This layer 304 may be made from a single coated tape having a layer of Adhesive 420®, made by 3M®. Exemplary tapes include single-sided variants of double-sided tapes having product nos. 9783, 9795, and 9795B, and available from 3M®. The laminate layer is typically 50-200μ thick, for example 125μ thick. Other acceptable layers may be made from adhesive tapes that utilize micro-capsule based adhesives.

The label 306 can be made from polypropylene or other plastic with pressure sensitive adhesive. The label 306 can be typically between about 50 and 150 microns thick. In some embodiments, the label 306 can be configured to seal the wax loading holes of the valves in the substrate 302. In some embodiments, the label 306 can trap air used for valve actuation. In some embodiments, the label 306 can serve as a location for operator markings. The label 306 can include identifying characteristics, such as a barcode number, lot number and expiry date of the cartridge. In some embodiments, the label 306 has a space and a writable surface that permits a user to make an identifying annotation on the label, by hand. The label 306 can be a single-piece layer, though it would be understood by one of ordinary skill in the art that the label 306 can be formed in two or more separate pieces.

The label 306 can be printed with various types of information, including but not limited to a manufacturer's logo, a part number, and index numbers for each of the sample lanes. In various embodiments, the label 306 includes a computer-readable or scan-able portion that may contain certain identifying indicia such as a lot number, expiry date, or a unique identifier. For example, the label 306 can include a bar code, a radio frequency tag, or one or more computer-readable, or optically scan-able, characters. The readable portion of the label 306 can be positioned such that it can be read by a sample identification verifier. The label 306 can include a cut-out 318 from an edge or a corner of the label 306.

In some embodiments, the microfluidic cartridge 300 further includes a registration member 316 that ensures that the cartridge is received by a complementary diagnostic apparatus in a single orientation, for example, in a receiving bay of the apparatus. The registration member 316 may be a cut-out from an edge or a corner of the cartridge (as shown in FIG. 3A), or may be a series of notches, wedge or curved-shaped cutouts, or some other configuration of shapes that require a unique orientation of placement in the apparatus.

In some embodiments, the microfluidic cartridge 300 has a size substantially the same as that of a 96-well plate as is customarily used in the art. Advantageously, then, the cartridge may be used with plate handlers used elsewhere in the art.

In some embodiments, the microfluidic cartridge 300 includes two or more positioning elements, or fiducials, for use when filling the valves with thermally responsive material. The positioning elements may be located on the substrate 302, typically the upper face thereof. In some embodiments, the fiducials can be on diagonally opposed corners of the substrate but are not limited to such positions.

As described herein, above each reaction chamber is a window 320 that permits optical detection, such as detection of fluorescence from a fluorescent substance, such as a fluorogenic hybridization probe, in a reaction chamber when a detector is situated above the window 320. The plurality of windows 320 can be formed in the label 306. The number of windows 320 can correspond to the number of reaction chambers (e.g., 1:1 such as 24 reaction chambers, 24 windows or 12 reaction chambers, 12 windows, etc.). Other configurations are contemplated for the windows 320, such as in shape, position, and/or number. In the illustrated embodiment, the windows 320 have an oblong shape. The windows 320 can have a surface area equal or greater than the area of the corresponding reaction chamber.

Embodiments of compressible pads according to the present technology will now be described. FIG. 4 illustrates a non-limiting example of the compressible pad 314 according to the present technology. The cartridge 300 can include the compressible pad 314. The compressible pad 314 can be formed of a material with a low compression force deflection, as described herein. The compressible pad 314 can be made of a material that easily compresses as described herein. The compressible pad 314 can be formed of a mechanically compliant material. For example, the mechanically compliant material of the compressible pad 314 can have a thickness of about 0.035″ (about 0.9 mm). Other thicknesses are suitable, e.g., approximately 0.5 mm, approximately 1 mm, approximately 1.5 mm, approximately 2 mm, between 0 mm and 1 mm, between 0.5 mm and 1.5 mm, between 1 mm and 2 mm, between 1.5 mm and 2.5 mm, between 0 mm and 2 mm, between 0.5 mm and 2.5 mm, between 1 mm and 3 mm, between 1.5 mm and 3.5 mm, etc.

In some embodiments, the compressible pad 314 is incorporated into the consumable, e.g., the microfluidic cartridge. In some embodiments, a compressible pad (not shown) is incorporated into the diagnostic instrument (e.g., into a detector that makes physical contact with the microfluidic cartridge during a detection procedure).

The compressible pad 314 can be a heat sealable layer and can be attached to the microfluidic cartridge using, for example, pressure sensitive adhesive. The compressible pad 314 can be compressible as described herein. The thickness of the compressible pad 314 can be from 0.1-2.5 mm at no compression, typically about 1.5 mm thick at no compression.

As described herein, the cartridge 300, and in particular the substrate 302, can include a registration member 316 that ensures that the cartridge is received by a complementary diagnostic apparatus in a single orientation, for example, in a receiving bay of the apparatus. The registration member 316 may be a cut-out from an edge or a corner of the cartridge (as shown in FIG. 3A), or may be a series of notches, wedge or curved-shaped cutouts, or some other configuration of shapes that require a unique orientation of placement in the apparatus. The compressible pad 314 can include a cut-out 322 from an edge or a corner of the compressible pad 314. The cut-out 322 can correspond to the cut-out 318 of the label 306 shown in FIG. 3A.

As described herein, above each reaction chamber is the window 320 in the label 306 that permits optical detection in a reaction chamber when a detector is situated above window 320. The compressible pad 314 can include a plurality of windows 324. The number of windows 324 can correspond to the number of reaction chambers (e.g., 1:1 such as 24 reaction chambers, 24 windows or 12 reaction chambers, 12 windows, etc.). Other configurations are contemplated for the windows 324, such as in shape, position, and/or number. In the illustrated embodiment, the windows 324 have an oblong or elongate shape. The windows 324 can have a surface area equal or greater than the area of the corresponding reaction chamber. The windows 324 can correspond in number and/or shape to the windows 320 of the label 306 shown in FIG. 3.

As described herein, the reaction chambers in adjacent sample lanes are staggered with respect to one another. In some embodiments, the sample inlets are all disposed along a single line parallel to the x-axis of the microfluidic cartridge 300. A reaction chamber in a first bank of sample lanes can be aligned with a reaction chamber in a second bank of sample lanes, wherein the reaction chambers are aligned transverse to the single line of sample inlets. In some embodiments, the 24-lane cartridge has two banks of twelve reaction chambers 326, 328. The first bank of twelve reaction chambers 326 is closer to the edge with the registration member 316. The second bank of twelve reaction chambers 328 is farther from the edge with the registration member 316. The reaction chambers can form a grid. Other configurations are contemplated.

In some embodiments, the 24-lane cartridge has two banks of twelve windows, formed from windows 320 in the label 306 and windows 324 in the compressible pad 314. The windows 320, 324 can form a grid. In some embodiments, a window 320 in the label 306 and a window 324 overlay each other to form a window pair, such that light can be transmitted therethrough. The surface area of each of the windows 320, 324 can be larger than the surface area of the corresponding reaction chamber. In the illustrated embodiment, each window pair 320, 324 encompasses the area around one reaction chamber. In another embodiment (not illustrated), each window pair 320, 324 encompasses the area around two or more reaction chambers. In still another embodiment (not illustrated), each window pair 320, 324 encompasses the area around a bank of reaction chambers. In this embodiment, the label 306 and the compressible pad 314 each include two window, a first window over the first bank 326 and a second window over the second bank 328. In a further embodiment (not illustrated), there is a single window pair 320, 324 that encompasses the area around all 24 reaction chambers of the cartridge 300. In this embodiment, there is a single window over all reaction chambers of the cartridge 300.

In some embodiments, the compressible pad 314 can be a separate layer that is coupled to the label 306. The label 306 and/or the compressible pad 314 can include an adhesive surface to couple the components together. Other methods of coupling are contemplated. FIG. 3A illustrates an embodiment wherein the compressible pad 314 is adhered to the top of a PCR cartridge 300, and the white cartridge label 306 is adhered on top of the compressible pad 314. The label 306 has been partially removed to show the compressible pad 314 below the label 306.

In some embodiments, the compressible pad 314 and the label 306 can be combined in a single layer. In some embodiments, the label 306 can be omitted. In such embodiments, the compressible pad 314 can include a top surface for displaying barcoding and manufacturing information, as described above. In some embodiments, the compressible pad 314 is a white or light color. In some embodiments, label information can be printed directly onto the compressible material, thereby eliminating the label 306. In some embodiments, omitting the label 306 can eliminate the possibility that delamination will between the compressible pad 314 and the label 306.

In some embodiments, a compressible pad 314 is a fully separable compressible pad. In some embodiments, a compressible pad 314 is a separate or independently formed component. The compressible pad 314 can be placed on the cartridge 300, such as a top surface 308 of the cartridge 300. In some embodiments, the compressible pad is applied on top of the label 306 of the cartridge 300 (this embodiment is not shown in FIG. 3A). In some embodiments, the compressible pad can be re-usable after completion of PCR amplification, for example by removing the compressible pad 314 from a first cartridge 300 and applying this same compressible pad 314 to a second cartridge 300. This embodiment shows similar improvements of thermal energy transfer to the reaction chambers as other embodiments disclosed herein. In some embodiments, such as those described herein, the compressible pad 314 is integrated onto or into the cartridge 300. For example, the compressible pad 314 may not be intended to be re-usable; disposal of the cartridge 300 after amplification of one or more samples also disposes of the compressible pad 314 that is integrated with the cartridge 300. The compressible pad 314 can be integrated into the construction of the cartridge 300. The compressible pad 314, when integrated, can reduce the risk of delamination during use.

In some embodiments, the cartridge 300 is disposable. After PCR has been carried out on a sample, and presence or absence of a polynucleotide of interest has been determined, it is typical that the amplified sample remains on the cartridge and that the cartridge is either used again (if one or more sample lanes remain open), or disposed of. Should a user wish to run a post amplification analysis, such as gel electrophoresis, the user may pierce a hole through the laminate 304 of the cartridge 300, and recover an amount—typically about 1.5 microliter—of PCR product. In one non-limiting embodiment, a user may place the individual sample lane on a special narrow heated plate, maintained at a temperature to melt wax in a valve of that sample lane, and then aspirate the reacted sample from the inlet hole of that sample lane.

The microfluidic cartridge 300 may also be stackable, such as for easy storage or transport, or may be configured to be received by a loading device, that holds a plurality of cartridges in close proximity to one another, but without being in contact with one another. In various embodiments, during transport and storage, the microfluidic cartridge can be further surrounded by a sealed pouch to reduce effects of, e.g., water vapor. The microfluidic cartridge can be sealed in the pouch with an inert gas. The microfluidic cartridge can be disposable, such as intended for a single use. The microfluidic cartridge can be disposable for example after one or more of its sample lanes have been used.

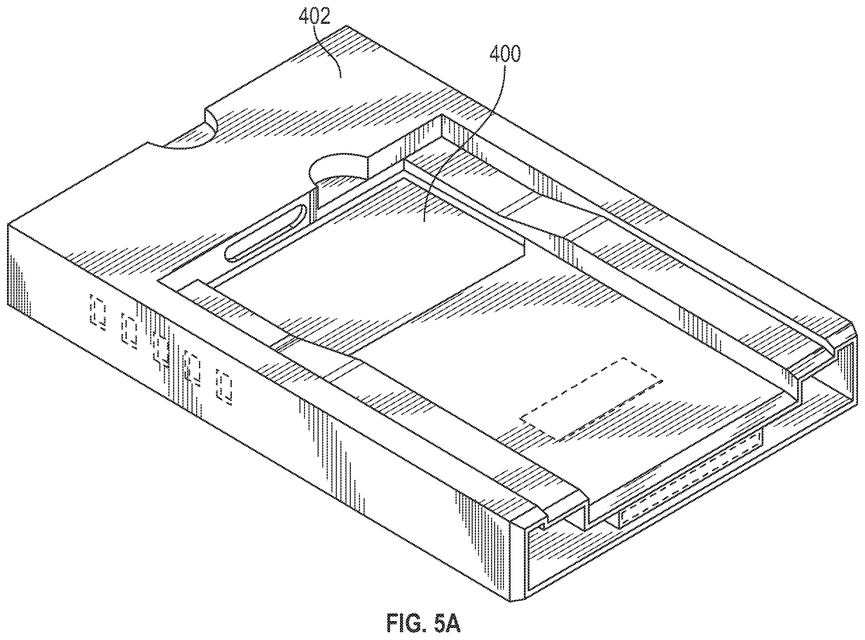

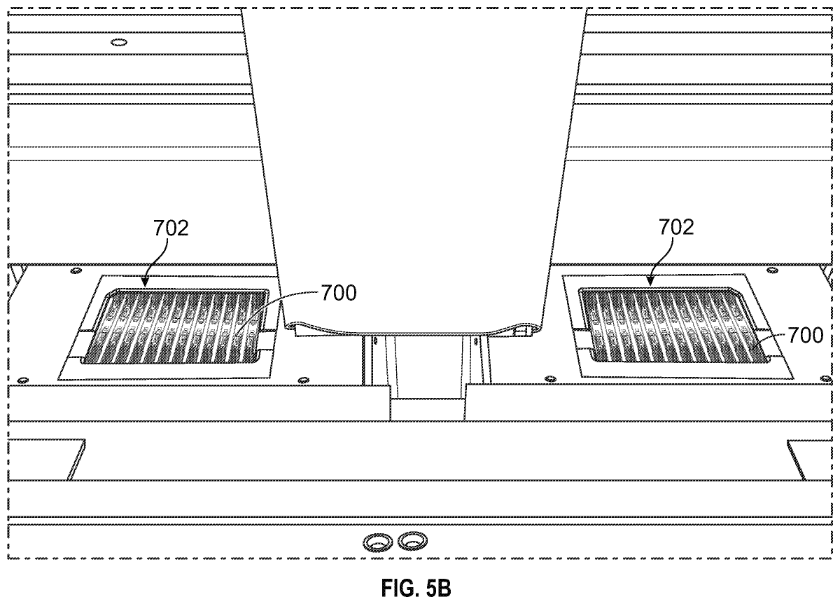

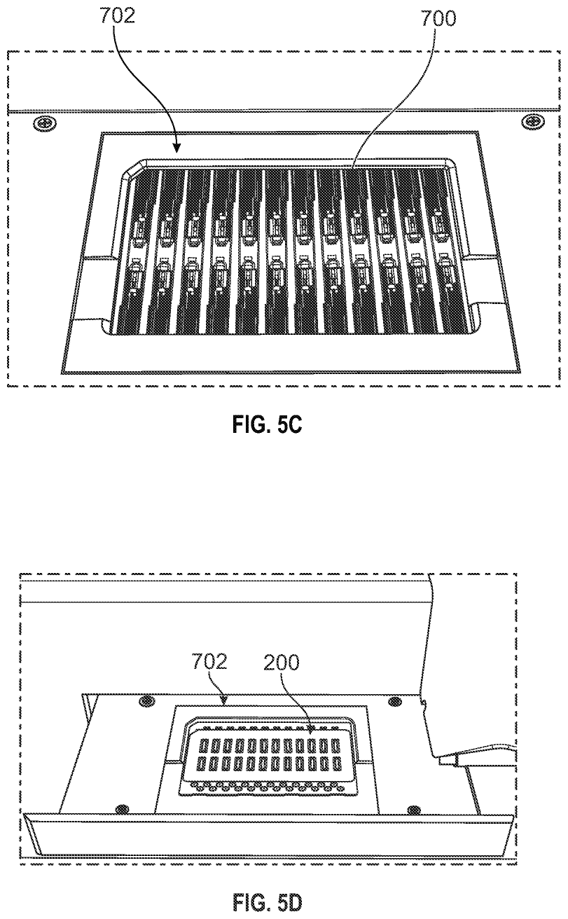

Non-limiting examples of heating assemblies according to the present technology will now be described in detail. FIG. 5A illustrates an example heater module 400 of a receiving bay 402. The heater module 400 can include a recessed surface that provides a platform for supporting a microfluidic cartridge in the receiving bay. In use, cartridge 300 is typically thermally associated with an array of heat sources configured to apply heat to various components of the device (e.g., reaction chamber). Exemplary such heater arrays including the heat sources are further described herein. Additional embodiments of heater arrays are described in U.S. patent application Ser. No. 11/940,315, entitled “Heater Unit for Microfluidic Diagnostic System” and filed on Nov. 14, 2007, the specification of which is incorporated herein by reference in its entirety.

FIG. 5B illustrates another example heater module 700 of a receiving bay 702. In this non-limiting embodiment, the system includes two receiving bays 702, each configured to receive a microfluidic cartridge of the present technology. FIG. 5C illustrates a close-up view of the heater module 700 of the left receiving bay 702. FIG. 5D illustrates a close-up view of the heater module 700 of FIG. 5C with a microfluidic cartridge 200 received in the receiving bay 702.

The microfluidic substrates described herein are configured to accept heat from a contact heat source, such as found in a heater unit. The heater unit typically comprises a heater board or heater chip that is configured to deliver heat to specific regions of the microfluidic substrate, including but not limited to one or more microfluidic components, at specific times. For example, the heat source is configured so that particular heating elements are situated adjacent to specific components of the microfluidic network on the substrate. In certain embodiments, the apparatus uniformly controls the heating of a region of a microfluidic network. In an exemplary embodiment, multiple heaters can be configured to simultaneously and uniformly heat a region, such as the PCR reaction chamber, of the microfluidic substrate.

Heaters are situated in a heater substrate layer directly under the microfluidic substrate. In non-limiting examples, heaters can be photolithographically defined and etched metal layers of gold (typically about 3,000 Å thick). Layers of 400 Å of TiW are deposited on top and bottom of the gold layer to serve as an adhesion layer. The substrate can be glass, fused silica or quartz wafer having a thickness of 0.4 mm, 0.5 mm, 0.7 mm, or 1 mm. A thin electrically-insulative layer of 2 μm silicon oxide serves as an insulative layer on top of the metal layer. Additional thin electrically insulative layers such as 2-4 g/m of Parylene may also be deposited on top of the silicon oxide surface.

An exemplary set of heaters configured to heat, cyclically, PCR reaction chamber can be provided. It is to be understood that heater configurations to actuate other regions of a microfluidic cartridge such as other gates, valves, and actuators (if present in the cartridge), may be designed and deployed according to similar principles to those governing the heaters described herein.

An exemplary reaction chamber in a microfluidic substrate, typically a chamber or channel having a volume, is configured with a long side and a short side, each with an associated heating element. A reaction chamber may also be referred to as a PCR reactor, herein, and the region of a cartridge in which the reaction chamber is situated may be called a zone. The heater substrate in this non-limiting example includes four heaters disposed along the sides of, and configured to heat, a given reaction chamber: long top heater, long bottom heater, short left heater, and short right heater. The small gap between long top heater and long bottom heater results in a negligible temperature gradient (less than 1° C. difference across the width of the reaction chamber at any point along the length of the reaction chamber) and therefore an effectively uniform temperature throughout the reaction chamber. The heaters on the short edges of the reaction chamber provide heat to counteract the gradient created by the two long heaters from the center of the reactor to the edge of the reactor.

It would be understood by one of ordinary skill in the art that still other configurations of one or more heater(s) situated about a reaction chamber are consistent with the methods and apparatus described herein. For example, a “long” side of the reaction zone can be configured to be heated by two or more heaters. Specific orientations and configurations of heaters are used to create uniform zones of heating even on substrates having poor thermal conductivity because the poor thermal conductivity of glass, or quartz, polyimide, FR4, ceramic, or fused silica substrates is utilized to help in the independent operation of various microfluidic components such as valves (if present in the cartridge) and independent operation of the various sample lanes. It would be further understood by one of ordinary skill in the art, that the principles underlying the configuration of heaters around a reaction zone are similarly applicable to the arrangement of heaters adjacent to other components of the microfluidic cartridge, such as actuators, valves, and gates (if present in the cartridge).

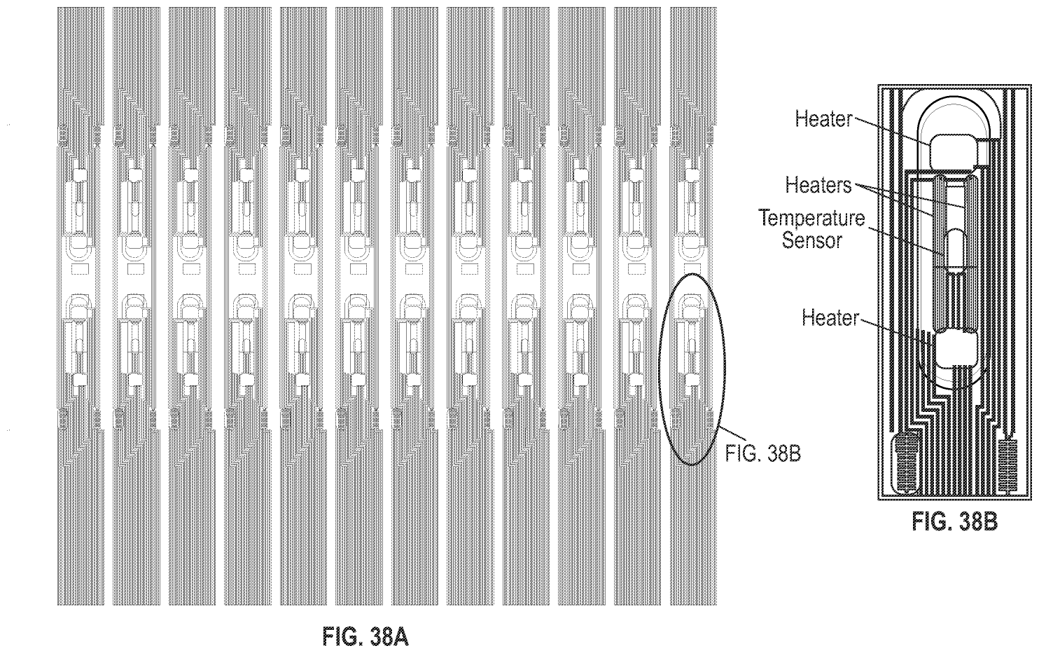

FIG. 38 illustrates a set of heater arrays of a heating apparatus configured to apply heat to microfluidic cartridges according to the present disclosure. For example, FIG. 38A illustrates a heater array configured to apply heat to a microfluidic cartridge that includes 24 sample lanes. FIG. 38B shows a blown-up view of one array configured to apply heat to one reaction chamber of a 24-sample lane cartridge, including heaters that carry current during operation and temperature sensors.

In some embodiments, the heat sources are controlled by a computer processor and actuated according to a desired protocol. Processors configured to operate microfluidic devices are described in, e.g., U.S. patent application Ser. No. 12/173,023, entitled “Integrated Apparatus for Performing Nucleic Acid Extraction and Diagnostic Testing on Multiple Biological Samples” and filed Jul. 14, 2008, which application is incorporated herein by reference. A processor, such as a microprocessor, is configured to control functions of various components of the system as shown, and is thereby in communication with each such component requiring control. It is to be understood that many such control functions can optionally be carried out manually, and not under control of the processor. Furthermore, the order in which the various functions are described, in the following, is not limiting upon the order in which the processor executes instructions when the apparatus is operating. Thus, processor can be configured to receive data about a sample to be analyzed, e.g., from a sample reader, which may be a barcode reader, an optical character reader, or an RFID scanner (radio frequency tag reader). It is also to be understood that, although a single processor is described as controlling all operations, but such operations may be distributed, as convenient, over more than one processor.

A processor can be configured to accept user instructions from an input, where such instructions may include instructions to start analyzing the sample, and choices of operating conditions. In various embodiments, the input can include one or more input devices, such as but not limited to: a keyboard, a touch-sensitive surface, a microphone, a track-pad, a retinal scanner, a holographic projection of an input device, and a mouse.

A processor can be also configured to communicate with a display, so that, for example, information about an analysis is transmitted to the display and thereby communicated to a user of the system. Such information includes but is not limited to: the current status of the apparatus; progress of PCR thermocycling; and a warning message in case of malfunction of either system or cartridge. Additionally, processor may transmit one or more questions to be displayed on display that prompt a user to provide input in response thereto. Thus, in certain embodiments, input and display are integrated with one another.

A processor can be optionally further configured to transmit results of an analysis to an output device such as a printer, a visual display, a display that utilizes a holographic projection, or a speaker, or a combination thereof.

A processor can be still further optionally connected via a communication interface such as a network interface to a computer network. The communication interface can be one or more interfaces selected from the group consisting of: a serial connection, a parallel connection, a wireless network connection, a USB connection, and a wired network connection. Thereby, when the system is suitably addressed on the network, a remote user may access the processor and transmit instructions, input data, or retrieve data, such as may be stored in a memory (not shown) associated with the processor, or on some other computer-readable medium that is in communication with the processor. The interface may also thereby permit extraction of data to a remote location, such as a personal computer, personal digital assistant, or network storage device such as computer server or disk farm. The apparatus may further be configured to permit a user to e-mail results of an analysis directly to some other party, such as a healthcare provider, or a diagnostic facility, or a patient.

Additionally, in various embodiments, the apparatus can further comprise a data storage medium configured to receive data from one or more of the processor, an input device, and a communication interface, the data storage medium being one or more media selected from the group consisting of: a hard disk drive, an optical disk drive, a flash card, and a CD-Rom.

A processor can be further configured to control various aspects of sample preparation and diagnosis, as follows in overview, and as further described in detail herein. The microfluidic cartridge 200, 300 is configured to operate in conjunction with a complementary rack (not shown). The rack is itself configured, as further described herein, to receive a number of biological samples in a form suitable for work-up and diagnostic analysis, and a number of holders that are equipped with various reagents, pipette tips and receptacles. The rack is configured so that, during sample work-up, samples are processed in the respective holders, the processing including being subjected, individually, to heating and cooling via a heater assembly. The heating functions of the heater assembly can be controlled by the processor. Heater assembly operates in conjunction with a separator, such as a magnetic separator, that also can be controlled by processor to move into and out of close proximity to one or more processing chambers associated with the holders, wherein particles such as magnetic particles are present.

Liquid dispenser (not shown), which similarly can be controlled by processor, is configured to carry out various suck and dispense operations on respective sample, fluids and reagents in the holders, to achieve extraction of nucleic acid from the samples. Liquid dispenser can carry out such operations on multiple holders simultaneously. Sample reader is configured to transmit identifying indicia about the sample, and in some instances the holder, to processor. In some embodiments a sample reader is attached to the liquid dispenser and can thereby read indicia about a sample above which the liquid dispenser is situated. In other embodiments the sample reader is not attached to the liquid dispenser and is independently movable, under control of the processor. Liquid dispenser is also configured to take aliquots of fluid containing nucleic acid extracted from one or more samples and direct them to a receiving bay in which a microfluidic cartridge 200, 300 is received. The receiving bay is in communication with a heater or a set of heaters that can be controlled by processor in such a way that specific regions of the cartridge are heated at specific times during analysis. Liquid dispenser is thus configured to take aliquots of fluid containing nucleic acid extracted from one or more samples and direct them to respective inlets in the microfluidic cartridge. Cartridge is configured to amplify, such as by carrying out PCR, on the respective nucleic acids. The processor is also configured to control a detector that receives an indication of a diagnosis from the cartridge. The diagnosis can be transmitted to the output device and/or the display, as described hereinabove.

A suitable processor can be designed and manufactured according to, respectively, design principles and semiconductor processing methods known in the art. In some embodiments, an apparatus includes a bay configured to selectively receive the microfluidic cartridge; at least one heat source thermally coupled to the bay; and coupled to a processor as further described herein, wherein the heat source is configured to heat individual sample lanes in the cartridge, and the processor is configured to control application of heat to the individual sample lanes, separately, in all simultaneously, or in groups simultaneously. In use, cartridge 200, 300 is typically thermally associated with an array of heat sources configured to operate the components (e.g., valves, gates, and processing region) of the device. In some embodiments, the heat sources are operated by an operating system, which operates the device during use. The operating system includes a processor (e.g., a computer) configured to actuate the heat sources according to a desired protocol. In some embodiments, temperature sensors are preferably configured to transmit information about temperature in their vicinity to the processor at such times as the heaters are not receiving current that causes them to heat. This can be achieved with appropriate control of current cycles.

As described herein, the application of pressure can facilitate contact between the microfluidic cartridge and heat sources of the heater array. In some embodiments, the pressure can be about 1 psi. The pressure is sufficient to enhance contact between the cartridge and the heat sources to assist in achieving better thermal contact between the heat sources and the heat-receivable parts of the cartridge. In some embodiments, the pressure can prevent the bottom laminate layer 304 from expanding, as would happen if the PCR channel was partially filled with liquid and the entrapped air is thermally expanded during thermocycling.

Each reaction chamber is heated through a series of cycles to carry out amplification of nucleotides in the sample according to an amplification protocol. The inside walls of the channel in the PCR reactor are typically made very smooth and polished to a shiny finish during manufacture. This is in order to minimize any microscopic quantities of air trapped in the surface of the PCR channel, which would cause bubbling during the thermocycling steps. The presence of bubbles especially in the detection region of the PCR channel could also cause a false or inaccurate reading while monitoring progress of the PCR.

Referring to FIG. 1A, the reaction chambers can have dimensions (such as a shallow depth) such that the temperature gradient across the depth of the channel is minimized. Referring to FIG. 2A, the reaction chambers are deeper and wider, for instance to accommodate larger samples for PCR. In the illustrative embodiment of FIG. 2A, the wider, deeper wells can require increased thermal contact between the cartridge and the heater substrate to ensure the temperature gradient across the depth of the channel is minimized, thereby ensuring optimal thermal uniformity and enhance PCR amplification. In some embodiments, the compressible pad 314 can allow for the use of wider, deeper wells by improving pressure distribution and therefore increasing contact between the microfluidic cartridge and the heater substrate.

In some embodiments, the area of the substrate 302 above the reaction chamber can be a thinned down section to reduce thermal mass and autofluorescence from plastic in the substrate. Also described herein, the label 306 can include windows 320 and the compressible pad 314 can include windows 324 to allow visualization of the reaction chambers and transmission of light to and from the reaction chambers. The design of the cartridge 300 can permit an optical detector to more reliably monitor progress of the reaction and also to detect fluorescence from a probe that binds to a quantity of amplified nucleotide. In some embodiments, a region of the substrate 302 can be made of thinner material than the rest of the substrate 302 so as to reduce glare, autofluorescence, and undue absorption of fluorescence.

As described herein, the microfluidic cartridges can be configured to be positioned in a complementary receiving bay in an apparatus that contains a heater unit. Non-limiting examples of heater units are illustrated in FIG. 5A and FIGS. 5B-5D. The heater unit is configured to deliver heat to specific regions of the cartridge, including but not limited to one or more reaction chambers, at specific times. In certain embodiments, the apparatus uniformly controls the heating of a region of a microfluidic network. In an exemplary embodiment, multiple heaters can be configured to simultaneously and uniformly heat a single region, such as the PCR reaction chamber, of the microfluidic cartridge. In other embodiments, portions of different sample lanes are heated simultaneously and independently of one another.

The microfluidic cartridge 300 can have a registration member 316 that fits into a complementary feature of the receiving bay. The registration member 316 can be, for example, a cut-out on an edge of the cartridge 300 and the receiving bay can include a complementary feature to the registration member 316. By selectively receiving the cartridge, the receiving bay can help the cartridge be placed in such a way that the apparatus can properly operate on the cartridge.

The receiving bay can also be configured so that heat sources of the apparatus that operate on the microfluidic cartridge 300 are positioned to properly operate thereon. For example, a contact heat source can be positioned in the receiving bay such that it can be thermally coupled to one or more distinct locations on a microfluidic cartridge 300 that is selectively received in the bay. Microheaters in the heater module as further described herein are aligned with corresponding heat-requiring microcomponents (such as valves, pumps, gates, reaction chambers, etc.). The microheaters, arranged in a set to deliver heat to a specific area of the cartridge 300, can be designed to be slightly bigger than the heat requiring microfluidic components so that even though the cartridge may be off-centered from the heater set, the individual components can still function effectively.

As further described elsewhere herein, the lower surface of the cartridge can have a layer of mechanically compliant heat transfer laminate 304 that can enable thermal contact between the microfluidic cartridge 300 and the heater substrate of the heater module. In some embodiments, as described herein, a minimal pressure, such as a pressure of 1 psi, can be employed for reliable operation of the reaction chambers present in the microfluidic cartridge.

Referring back to FIG. 3, the PCR reaction chamber (for example, a reaction chamber of 150μ deep×700μ wide), is shown in the substrate layer 302 of the cartridge 300. The laminate layer 304 of the cartridge (for example, 125μ thick) is directly under the PCR reaction chamber. In some embodiments, a region of the substrate 302 can be made of thinner material than the rest of the substrate 302 so as to permit the PCR reaction chamber to be more responsive to a heating cycle (for example, to rapidly heat and cool between temperatures appropriate for denaturing and annealing steps). Heaters are situated in a heater module directly under the laminate layer 304 when the cartridge is received by the heater module.

In some embodiments, each reaction chamber is configured with a long side and a short side. Each of the sides corresponds to an associated heating element located in the heater substrate. The heater substrate therefore includes four heaters disposed along the sides of, and configured to heat, the PCR reaction chamber: long top heater, long bottom heater, short left heater, and short right heater. In some embodiments, the small gap between long top heater and long bottom heater results in a negligible temperature gradient (less than 1° C. difference across the width of the PCR channel at any point along the length of the PCR reaction chamber) and therefore an effectively uniform temperature throughout the PCR reaction chamber. The heaters on the short edges of the PCR reactor provide heat to counteract the gradient created by the two long heaters from the center of the reactor to the edge of the reactor. It would be understood by one of ordinary skill in the art that still other configurations of one or more heater(s) situated about a PCR reaction chamber are consistent with the methods and apparatus described herein. For example, a ‘long’ side of the reaction chamber can be configured to be heated by two or more heaters.

The heat source can be, for example, a resistive heater or network of resistive heaters. In some embodiments, the at least one heat source can be a contact heat source selected from a resistive heater (or network thereof), a radiator, a fluidic heat exchanger and a Peltier device. The contact heat source can be configured at the receiving bay to be thermally coupled to one or more distinct locations of a microfluidic cartridge received in the receiving bay, whereby the distinct locations are selectively heated. The contact heat source typically includes a plurality of contact heat sources, each configured at the receiving bay to be independently thermally coupled to a different distinct location in a microfluidic cartridge received therein, whereby the distinct locations are independently heated. The contact heat sources can be configured to be in direct physical contact with one or more distinct locations of a microfluidic cartridge received in the bay. In various embodiments, each contact source heater can be configured to heat a distinct location having an average diameter in 2 dimensions from about 1 millimeter (mm) to about 15 mm (typically about 1 mm to about 10 mm), or a distinct location having a surface area of between about 1 mm² about 225 mm² (typically between about 1 mm² and about 100 mm², or in some embodiments between about 5 mm² and about 50 mm²). Various configurations of heat sources are further described in U.S. patent application Ser. No. 11/940,315, entitled “Heater Unit for Microfluidic Diagnostic System” and filed on Nov. 14, 2017, which is incorporated by reference in its entirety.

In some embodiments, the heaters are photolithographically defined and etched metal layers of gold (typically about 3,000 Å thick). Layers of 400 Å of TiW can be deposited on top and bottom of the gold layer to serve as an adhesion layer. In some embodiments, the heater substrate is glass, fused silica or a quartz wafer having a thickness of 0.4 mm, 0.5 mm, 0.7 mm, or 1 mm. In some embodiments, a thin electrically-insulative layer of 2 μm silicon oxide serves as an insulative layer on top of the metal layer. In some embodiments, additional thin electrically insulative layers such as 2-4 μm of Parylene may also be deposited on top of the silicon oxide surface. In some embodiments, two long heaters and two short heaters run alongside and enclose an area that corresponds to each PCR reaction chamber. An exemplary heater array is described in U.S. patent application Ser. No. 11/940,315, entitled “Heater Unit for Microfluidic Diagnostic System” and filed on Nov. 14, 2017, the specification of which is incorporated herein by reference in its entirety.

Specific orientations and configurations of heaters are used to create uniform zones of heating even on substrates having poor thermal conductivity. The heater substrate can be formed of various materials, including glass, or quartz, polyimide, FR4, ceramic, or fused silica substrates. The heater module is utilized to help in the independent operation of various microfluidic components such as PCR reaction chambers and independent operation of the various sample lanes. The configuration for uniform heating for a single PCR reaction chamber can be applied to a multi-lane PCR cartridge in which multiple independent PCR reactions occur. In other embodiments, as further described in U.S. patent application Ser. No. 11/940,315, entitled “Heater Unit for Microfluidic Diagnostic System” and filed on Nov. 14, 2007, the heaters may have an associated temperature sensor, or may themselves function as sensors.

Generally, the heating of microfluidic components, such as a PCR reaction chamber, is controlled by passing currents through suitably configured microfabricated heaters. Under control of suitable circuitry, the sample lanes of a multi-lane cartridge can then be controlled independently of one another. This can lead to a greater energy efficiency of the apparatus, because not all heaters are heating at the same time, and a given heater is receiving current for only that fraction of the time when it is required to heat. Control systems and methods of controllably heating various heating elements are further described in U.S. patent application Ser. No. 11/940,315, entitled “Heater Unit for Microfluidic Diagnostic System” and filed on Nov. 14, 2007.

An example of thermal cycling performance in a PCR reaction chamber obtained with a configuration as described herein can include a protocol that is set to heat up the reaction mixture to 92° C., and maintain the temperature for 1 second, then cool to 62° C., and stay for 10 seconds. The cycle time shown is about 29 seconds, with 8 seconds required to heat from 62° C. and stabilize at 92° C., and 10 seconds required to cool from 92° C., and stabilize at 62° C. To minimize the overall time required for a PCR effective to produce detectable quantities of amplified material, it is important to minimize the time required for each cycle. Cycle times in the range 15-30 seconds, such as 18-25 seconds, and 20-22 seconds, are desirable. In general, an average PCR cycle time of 25 seconds as well as cycle times as low as 20 seconds are typical with the technology described herein. In some non-limiting examples, using reaction volumes less than a microliter (such as a few hundred nanoliters or less) permits use of an associated smaller PCR chamber, and enables cycle times as low as 15 seconds.

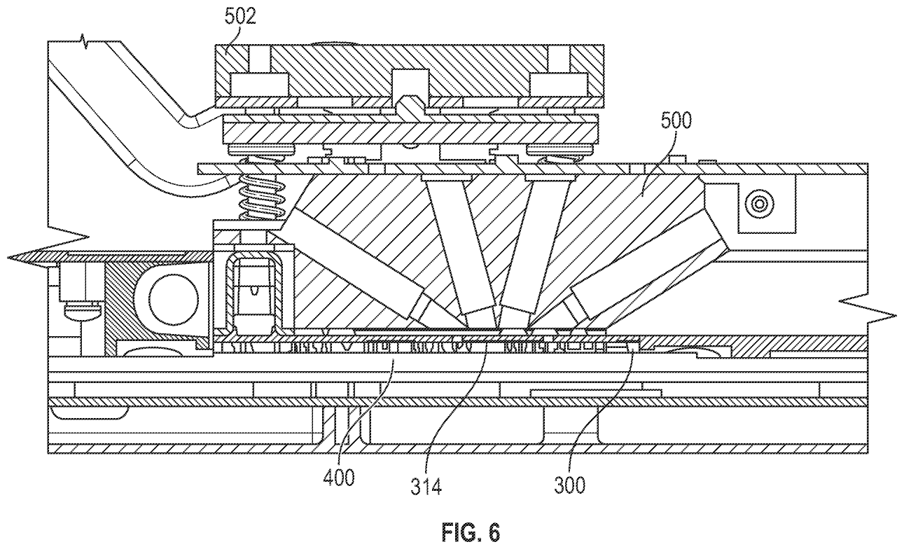

Non-limiting examples of optical detectors suitable for use with microfluidic cartridges of the present technology will now be described. Referring to FIG. 6, an embodiment of an optical detector 500 is illustrated. As described above, the heater module 400 is disposed under the microfluidic cartridge 300. In some embodiments, a thermally conductive, mechanically compliant layer such as the compressible pad 314 can lay at an interface between the microfluidic cartridge 300 and the optical detector 500. Typically, the microfluidic cartridge 300 and the heater module 400 can be planar at their respective interface surfaces, e.g., planar within about 100 microns, or more typically within about 25 microns. The compressible pad 314 can improve thermal coupling between microfluidic cartridge 300 and the heater module 400. Optical detector 500 can be disposed over the top surface of the microfluidic cartridge 300.

In various embodiments, the apparatus can further include one or more force members configured to apply force to at least a portion of a microfluidic cartridge 300 received in the receiving bay 402 comprising one or more heat sources. In the non-limiting embodiment of FIG. 6 shows, the force member includes a lever assembly 502 associated with the optical detector 500. In some embodiments, the system relies on pressure to be applied to the cartridge 300. A bottom surface of optical detector 500 can be made flat (e.g., within 250 microns, typically within 100 microns, more typically within 25 microns), and the bottom surface can press upon the cartridge 300. The cartridge 300 can include the compressible pad 314. Consequently, the optical detector 500 can compress the cartridge 300 thereby making the pressure, and thus the thermal contact with an underlying heater substrate of the heater module 400, more or less uniform over microfluidic cartridge 300.

It will be understood that the present technology is not limited to an optical detector including a lever assembly 502. Other force members can be suitably implemented. In one example, an automated platform including the optical detector 500 is lowered onto and pressed onto the microfluidic cartridge 300, where the microfluidic cartridge 300 is received in a receiving bay that remains stationary. Movement of the automated platform can be controlled by a processor of the diagnostic apparatus. In another example, an automated platform including the receiving bay 402 (and the microfluidic cartridge 300) is raised up and pressed into the bottom surface of the optical detector 500, where the optical detector 500 remains stationary. Movement of the automated platform can be controlled by a processor of the diagnostic apparatus.

Accordingly, embodiments of the diagnostic apparatus according to the present technology are configured to apply force to thermally couple the at least one heat source to at least a portion of the microfluidic cartridge 300. The application of force is important to ensure consistent thermal contact between the heater module 400 and the PCR reaction chamber in the microfluidic cartridge 300. In some embodiments, the lever assembly 502, similar mechanical force member, or automated platform can deliver a force (e.g., from 5-500 N, typically about 200-250 N) to generate a pressure (e.g., 2 psi) over the top or a portion of the top of microfluidic cartridge 300. In the embodiments in which the optical detector 500 moves above a stationary receiving bay 402, mechanical features of the optical detector 500 can press down on the microfluidic cartridge 300 after the optical detector 500 is in position, causing the reaction chambers to be in better thermal contact with the heater module 400. Positioning the optical detector 500 can thus apply a pressure to the cartridge 300. In the embodiments in which the receiving bay 402 moves below a stationary optical detector 500, mechanical features of the receiving bay 402 can press up into the microfluidic cartridge 300 after the receiving bay 402 is in position, causing the reaction chambers to be in better thermal contact with the heater module 400. Positioning the receiving bay 402 can thus apply a pressure to the cartridge 300.

Other configurations of applying pressure to the cartridge 300 to improve temperature uniformity and PCR efficiency are contemplated, including applying pressure with another component of the diagnostic instrument. In the illustrated embodiment, pressure is applied to the top surface of the cartridge 300 and the heater module 400 is placed below the cartridge 300, however, other configurations are contemplated.

The optical detector 500 can include a light source that selectively emits light in an absorption band of a fluorescent dye, and a light detector that selectively detects light in an emission band of the fluorescent dye, wherein the fluorescent dye corresponds to a fluorescent polynucleotide probe or a fragment thereof. Alternatively, for example, the optical detector 500 can include a bandpass-filtered diode that selectively emits light in the absorption band of the fluorescent dye and a bandpass filtered photodiode that selectively detects light in the emission band of the fluorescent dye. The optical detector 500 can be configured to independently detect a plurality of fluorescent dyes having different fluorescent emission spectra, wherein each fluorescent dye corresponds to a fluorescent polynucleotide probe or a fragment thereof. The optical detector 500 can be configured to independently detect a plurality of fluorescent dyes at a plurality of different locations of the microfluidic cartridge, wherein each fluorescent dye corresponds to a fluorescent polynucleotide probe or a fragment thereof in a different sample. The optical detector 500 can also be configured to detect the presence or absence of an analyte of interest in a sample in a PCR reaction chamber in a given sample lane, and to condition initiation of thermocycling upon affirmative detection of presence of the sample. In some embodiments, a cartridge and apparatus are configured so that the read-head of the optical detector 500 does not cover the sample inlets, thereby permitting loading of separate samples while other samples are undergoing PCR thermocycling. Further description of suitably configured detectors are described in U.S. patent application Ser. No. 11/940,321, entitled “Fluorescence Detector for Microfluidic Diagnostic System” and filed on Nov. 14, 2007, the entirety of which is incorporated herein by reference. The present technology provides for a fluorescent detector that is configured to detect light emitted for a probe characteristic of a polynucleotide. The polynucleotide is undergoing amplification in a microfluidic channel with which the detector is in optical communication. The detector is configured to detect minute quantities of polynucleotide, such as would be contained in a microfluidic volume. The detector can also be multiplexed to permit multiple concurrent measurements on multiple polynucleotides concurrently.

Although the various depictions herein describe a heater substrate disposed underneath a microfluidic cartridge, and a detector disposed on top of the microfluidic cartridge, it would be understood that an inverted arrangement would work equally as well. In such an embodiment, the heater would be forced down onto the microfluidic substrate, making contact therewith, and the detector would be mounted underneath the substrate, disposed to emit light upwards toward the microfluidic cartridge and to collect light exiting the microfluidic cartridge downwards towards the detector.

The compressible pad 314 can provide many advantages as described herein. The compressible pad 314 for the microfluidic cartridge 300 can be designed to improve pressure distribution, for instance, to improve the distribution of pressure of the bottom surface of the detector over the top surface of the microfluidic cartridge and, consequently, the distribution of pressure applied across the bottom surface of the microfluidic cartridge by the receiving bay. The compressible pad 314 for the microfluidic cartridge 300 can be designed to increase thermal uniformity, for instance, to improve uniform contact between the cartridge and the heater module. The compressible pad 314 for the microfluidic cartridge 300 can be designed to enhance PCR amplification, for instance, by facilitating the uniform application of heat to wider and/or deeper reaction chambers.

As described above, in some embodiments, the compressible pad 314 is adhered on top of the microfluidic cartridge 300. The cartridge 300 can include a substrate 302 made of cyclo-olefin polymer (COP) as described herein. The cartridge 300 can include twenty-four microfluidic reaction chambers configured to contain molecular material for PCR amplification. PCR amplification requires heating and cooling the fluid in each reaction chamber to specific temperatures in given amounts of time. In use, the cartridge 300 is placed on top of the heater substrate. In some embodiments, the heater substrate is a surface with heaters underneath. In use, a compressive load is applied to tightly hold the cartridge 300 between the heater module 400 and the optical detector 500. The optical detector 500 can include a rigid surface, including a rigid metal surface. The compressive load applied in embodiments of the present technology ensures physical contact between the cartridge 300 and the heater module 400, including an optimally-distributed physical contact between the cartridge 300 and the heater module 400. In some embodiments, heat is transferred from the heaters to the fluid in the cartridge 300 via thermal conduction or direct heater contact.

Due to surface roughness, mechanical variation, and/or inherent material irregularities, the rigid surfaces of the microfluidic cartridge 300 and the rigid surface of the heater module 400 that are brought together are unable to provide sufficient flatness for optimal contact with one another. In some embodiments, the compressible pad 314 includes a highly compressible material that is adhered on top of the cartridge 300. The compressible pad 314 can improve contact between the two rigid surfaces by introducing an element of compliance into an otherwise rigid system. The compressibility of the material of the compressible pad 314 allows for some areas to compress different amounts than others. This differential compression accommodates the inherent mechanical and material surface variations in the two surfaces, and result in a much more uniform pressure distribution across the entire cartridge 300. The compliant pad 314 enables a more uniform contact between the cartridge 300 and the heater module 400, thus providing more thorough and consistent heat transfer to each of the microfluidic reaction chambers.

The compressible pad 314 allows for more uniform physical contact and pressure distribution between the cartridge 300 and the heater module 400. This is advantageous because the uniform pressure results in fewer thermal losses, and more heat is able to be directly transferred to the cartridge 300. This advantageously improves the uniformity of heating and directly impacts the success and consistency of PCR amplification in embodiments of the present technology.

The heaters in the heater module 400 provide thermal conduction to fluid samples received in the cartridge 300. Upon compressing a cartridge 300 without a compressible pad against the heater module 400, there are some areas that make less contact than others because of mechanical and material surface variations, and inherent curvature and bowing in the heater surface and/or the surface of the microfluidic cartridge. This results in uneven pressure distribution across the cartridge 300. In use, areas with poorer physical contact between the heaters and the cartridge 300 will experience thermal losses. Therefore, less heat is delivered to the reaction chambers with poorer physical contact. This results in delays and inconsistencies to PCR amplification. The inconsistent physical contact can introduce significant variability in the performance of the overall assay.

Embodiments of the compressible pad 314 according to the present technology allow for some areas of the pad to compress more than others. This compressibility accommodates the inherent mechanical and material surface variations in the system, and ensures that all areas of the cartridge 300 have a more even pressure distribution. The compressible pad 314 can improve physical contact between the cartridge 300 and the heaters in the heater module 400, reduce thermal losses, and/or result in better PCR performance.

As described above, the compressible pad 314 can be incorporated directly into the label 306. The label 306 can include a top surface for displaying barcoding and manufacturing information, as well as other types of information. The label 306 is made of a thin polyester facestock material, which does not have any inherent compliance. In some embodiments, the compressible pad 314 is integrated directly into the existing label construction. There are various methods to accomplish this. In a first non-limiting example, the label 306 can include an adhesive lower surface which can bind to the compressible pad 314 to form an integrated label-pad structure. In a second non-limiting example, label information is applied directly onto the compressible material and the polyester facestock of the label 306 is eliminated entirely. In some embodiments, integrating the compliant material directly into the existing label can reduce delamination when compared to other embodiments and may be easily introduced into the existing manufacturing process and supply chain systems. FIG. 3 illustrates an embodiment of the first non-limiting example described above, wherein the compressible pad 314 is adhered to the top of a PCR cartridge 300, and the white cartridge label 306 is adhered on top of the compressible pad 314. The label 306 has been peeled back so that the construction can be easily viewed.

In some embodiments, a compressible pad 314 is a fully separate compressible pad and does not form an integral portion of the final manufactured microfluidic cartridge. The compressible pad 314 can be reversibly placed in contact with the cartridge 300, such as on a top surface 308 of the cartridge 300. In some embodiments, the compressible pad is applied on top of the label 306 of the cartridge 300 (this embodiment is not shown in FIG. 3A). In some embodiments, the compressible pad can be re-usable after completion of PCR amplification, for application onto another cartridge 300. This embodiment shows similar improvements of thermal energy transfer to the reaction chambers as other embodiments disclosed herein.

In some embodiments, a compressible pad is applied to the optical reader or a surface thereof instead of the cartridge (embodiment not shown). One benefit of this embodiment is that the compressible pad is no longer a part of the microfluidic cartridge. As described herein, in some embodiments, the microfluidic cartridge is disposable. In this embodiment, the compressible pad does not form a portion of a disposable microfluidic cartridge, but instead becomes a permanent part of the instrument (where it is re-used multiple times as each cartridge is used and disposed). The compressible pad in this embodiment can produce significant costs savings due to the reusability of the pad. In some embodiments, the optical detector 500 may be redesigned or altered to accommodate a re-usable compressible pad. In some cases, the compressible pad of this example is replaced after a particular number of uses, or after a particular amount of time. Regularly replacing the compressible pad in this manner can ensure that the pad incorporated in the instrument has optimal compression characteristics.

As described herein, the thermal uniformity across the cartridge 300 is dependent on the physical contact between the cartridge and a surface of the heater module 400. In some embodiments, heat transfer to the cartridge 300 can rely on direct conduction. As described herein, there are inherent surface irregularities, curvature, and mechanical variations in one or more of the heater substrate and the cartridge, the two surfaces may be unable to provide sufficient flatness for optimal contact with one another. Advantageously, embodiments of the present technology include a compressible pad 314 that incorporates a material with an extremely low compression force deflection. The compression force deflection is the amount of force it takes to compress the material by a given distance. Materials with lower compression force deflection compress more easily. Because embodiments of compressible pads according to the present the invention are highly compressible, different parts of the heater surface, microfluidic cartridge, and optical detector can compress by slightly different amounts, depending on when these components make contact with the other components. For example, the compressible pad 314 can allow different parts of the heater module 400 and/or the cartridge 300 to compress by slightly different amounts, depending on when and where the contact between the surfaces first occurs. The compressible pad 314 can introduce a level of flexibility into an otherwise rigid system. The compressible pad 314 can adjust for any inherent variation in the overall system. The compressible pad 314 therefore can improve the pressure distribution across the entire cartridge 300. The compressible pad 314 therefore can help ensure that all of the twenty-four reaction chambers make not just sufficient contact with the heaters of the heater module 400, but optimal contact with the heaters of the heater module 400. This improved thermal uniformity can make PCR amplification more consistent, reduce variation, and improve performance of the assay overall.

As described herein, two methods to determine characteristics of a material include durometer testing and compression force deflection testing. These methods are useful for determining the relative hardness or firmness of a material. Durometer testing is useful for measuring the hardness of a solid material, for instance solid material has a range of hardness. Compression Force Deflection (CFD) testing can be useful for measuring foam, spongy, or other non-firm materials. Both types of measurements are based on ASTM guidelines and methods which are incorporated by reference herein in their entirety.

Durometer testing utilizes a Shore harness scale, for example, Shore A. The Shore scales correlate with the testing apparatus that is utilized, in particular, the configuration of the testing indenter that contacts the material. The indenter applies a load to a small contact point on the material. Durometer testing assumes that the surface of the material is relatively uniform in hardness relative to the tested contact point. Different Shore scales are typically used for different material types, such as different materials with different hardness. As one example, Shore A is typically useful for softer elastomeric materials and Shore D is typically useful for harder elastomeric materials.

Compression Force Deflection testing, in contrast, compresses an entire material sample, wherein the sample is typically about 10 cm. The method involves determining the amount of stress at different levels of strain. The method allows for a determination of hardness or firmness at different compression levels. Compared with Durometer testing, Compression Force Deflection testing allows for a larger test sample, and the larger sample can facilitate a more accurate measurement of the characteristics of the material.

In embodiments of the present technology, the inventors discovered that durometer testing is typically less accurate than Compression Force Deflection testing for determining the hardness of the compressible pad 314, and consequently for assessing the suitability of particular compressible pad materials to achieve improved PCR test results. The compressible pad 314 comprises a compressible material, such as those described herein. The hardness of these materials can be dependent on compression level. The hardness of these materials can be dependent on the test area and can vary from test area to test area. As described herein, the indenters for durometer testing measure only a small point on the material, covering a small area of the overall surface of the material. This small point may not be representative of the larger sample, depending on the material of the compressible pad 314. In contrast, compression force deflection testing determines an average firmness for a larger sample size. Compression force deflection testing can determine the hardness of a material based on compression level typical of the designed application. For instance, compression force deflection testing can determine the hardness for a material based on the compression levels typical of a testing apparatus, and in some embodiments, the compression levels of the optical detector 500, designed to compress the compressible pad 314. As described herein, compression force deflection testing can be a more representative measurement of how the compressible pad 314 will perform when applied to the microfluidic cartridge 300.

### EXAMPLE

Having generally described embodiments of the present technology, a further understanding can be obtained by reference to certain specific examples which are provided herein for purposes of illustration only, and are not intended to be limiting.

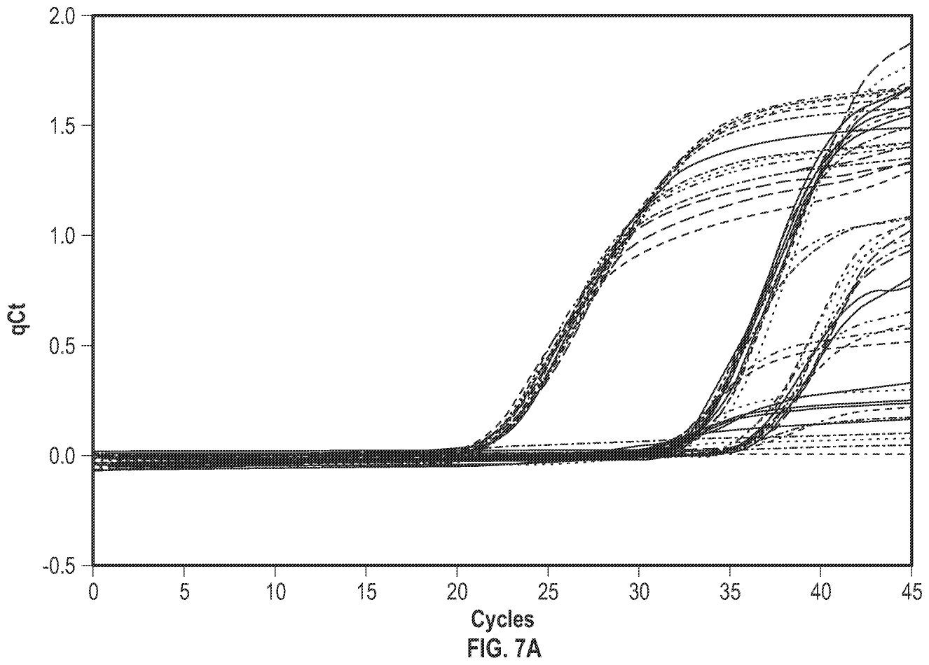

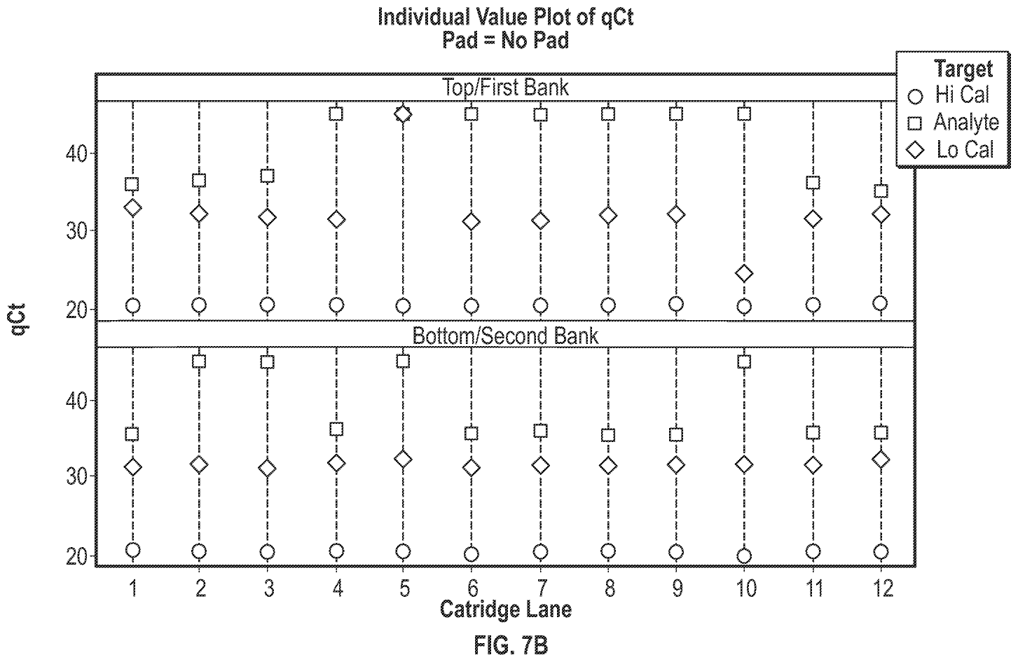

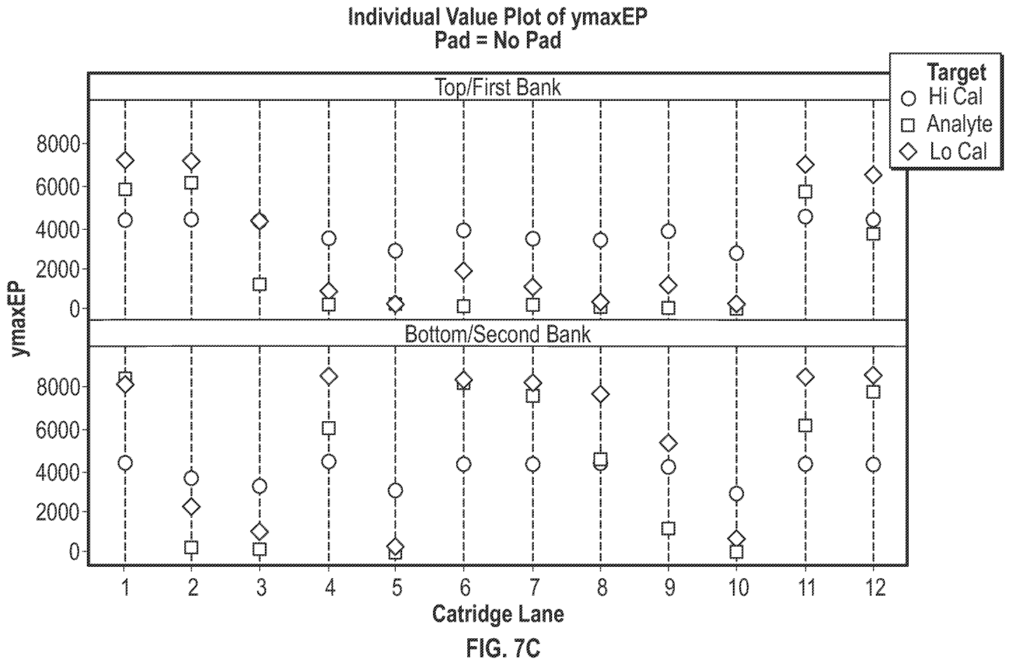

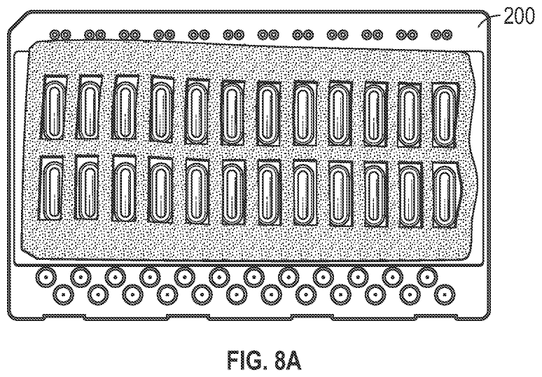

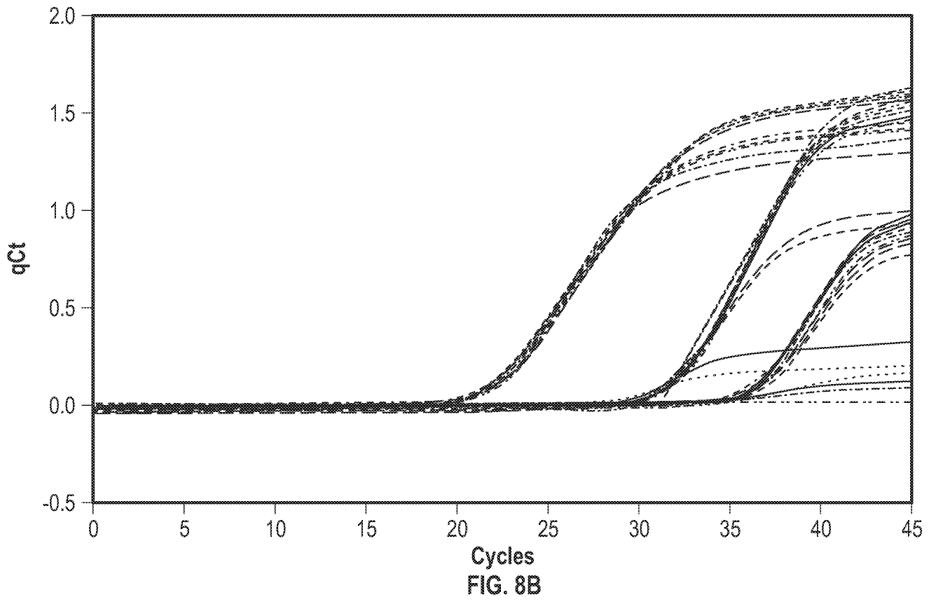

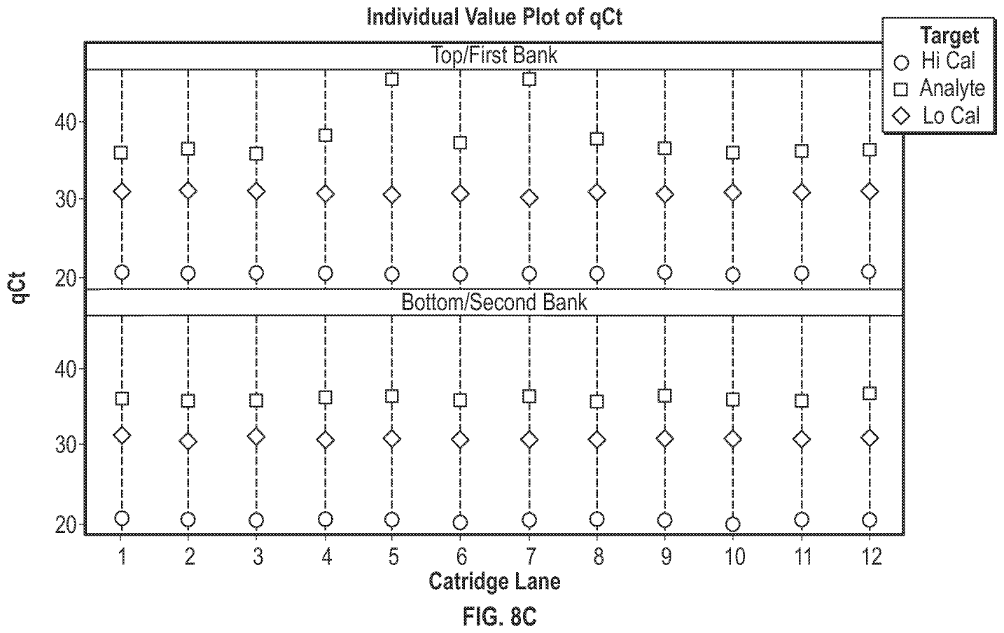

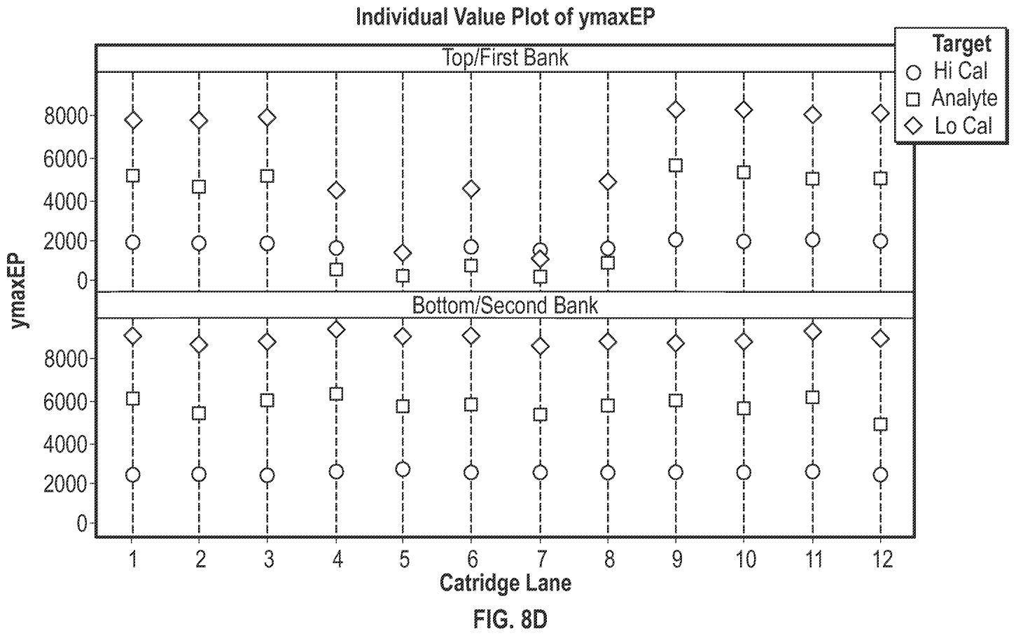

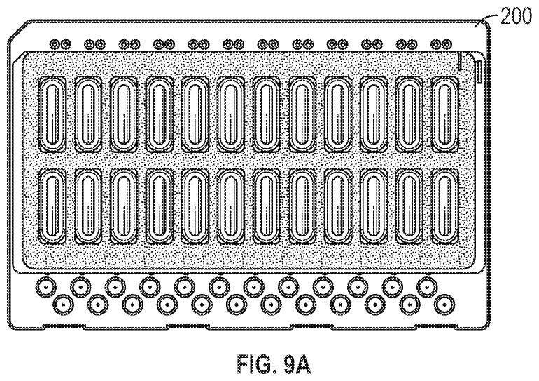

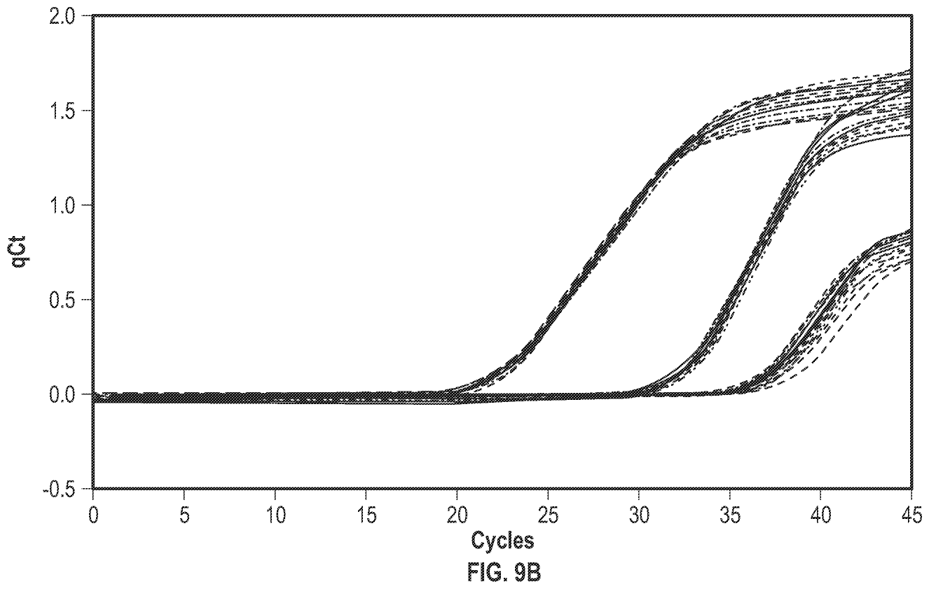

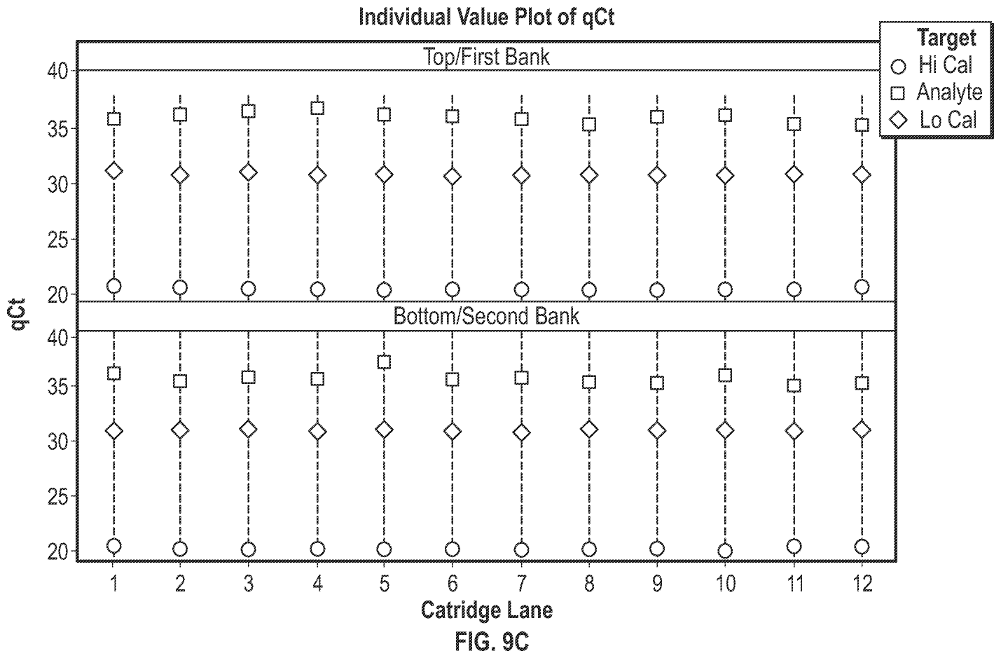

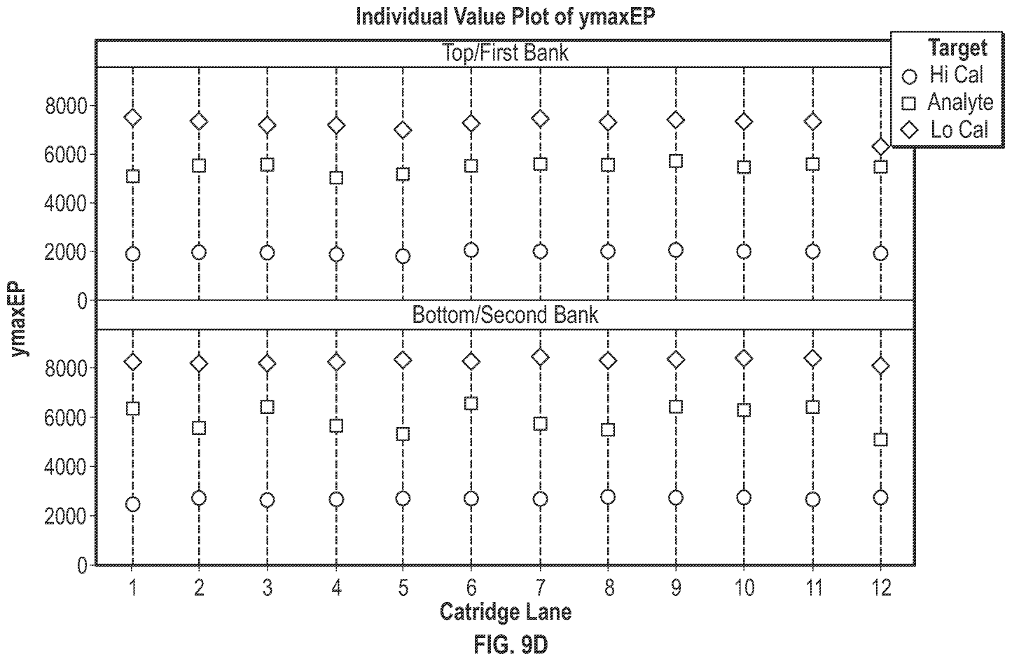

This example describes the identification of materials for the compressible pad 314. FIGS. 7A-7C show results for assay testing for an analyte of interest without a compressible pad. FIGS. 8A-8D show results for assay testing for the analyte of interest with a low durometer silicone compressible pad. FIGS. 9A-9D show results for assay testing for the analyte of interest with a PORON® foam compressible pad.

As described herein, the microfluidic cartridges 100, 200, 300 can be used to perform amplification protocols on samples that have been prepared to detect the presence or absence of many different types of analytes of interest. Embodiments of an automated molecular diagnostic test system described herein can prepare a specimen according to an analyte-specific assay to obtain a PCR-ready sample that is introduced into a microfluidic cartridge that is received in the system. One example analyte-specific test includes an assay test for a viral analyte of interest. The testing relates to detection of the viral analyte of interest using a molecular viral load assay. The assay is a real-time RT-PCR assay to quantify the amount of viral analyte of interest (“viral load”) in a sample. As described above, the assay can be performed on an automated molecular diagnostic test system of the present technology. Viral load is a numerical expression of the quantity of virus in a given volume. It can be expressed as viral particles per mL. A higher viral load correlates with a more severe viral infection. The quantity of virus/mL can be calculated by estimating the live amount of virus in a fluid specimen taken from a patient. For example, viral load can be given in RNA copies per milliliter of blood plasma. The assay can be used to track viral load during antiretroviral therapy, thereby allowing caregivers to measure and assess changes in the amount of the viral analyte during treatment.

The assay of this example can use two different RNA calibrator sequences, Hi Cal and Lo Cal. An RNA calibrator sequence (“calibrator”) is a synthetic RNA transcript of known sequence and quantity that is used to adjust the output of the assay measurement. This is in contrast to a “control,” a standard sample that can be included in the assay to assess the validity of the test result (rather than to adjust the output of the test result). The calibrator is designed to bind to a molecule with a complementary base sequence, also known as a probe. This process of specific binding is called hybridization. During sample preparation, a known quantity of calibrator is mixed with the patient specimen and PCR reagents. The prepared sample is amplified to detect and quantify target nucleic acids (viral analyte of interest) in the sample. The degree of hybridization between the calibrator and its corresponding probe is used to normalize measurements of the target nucleic acid with its corresponding probe. The calibrator is designed to amplify with the same efficiency as the target nucleic acid and to respond similarly to sources of variation (such as instrument and matrix variances). In the following examples, testing was performed to measure a quantity (as indicated by a qCt measurement) and assess other characteristics (for example, y max EP) of the following targets: a Hi Cal calibrator, a viral analyte of interest, and a Lo Cal calibrator in test samples.

Quantification of the target nucleic acid in the sample relies on a relationship between fluorescence and the number of amplification cycles on a logarithmic scale. The number of cycles at which the fluorescence exceeds a given detection threshold is sometimes referred to as the cycle threshold (Cₜ). During amplification, the quantity of the target nucleic acid doubles every cycle. So, for example, a sample whose cycle threshold precedes that of another sample by 3 cycles contained 2³=8 times more target nucleic acid. In the following assay testing, two perimeters were tested. The first parameter is a qCt score which indicates the first amplification cycle in which fluorescence is detected in a thermal cycling protocol including a plurality of amplification cycles. The second parameter is an y max EP score which indicates a maximum fluorescence unit in a final resting amplitude after a plurality of amplification cycles.

Optimal sample volumes for embodiments of a viral load assay test for a viral analyte of interest described herein are in the range of about 25 μL (rather than about 4 μL). As described above, such sample volumes can be obtained using wider, deeper reaction chamber in a thicker version of microfluidic cartridges of the present technology (thickness of about 1.68 mm for a cartridge with wider, deeper wells versus a cartridge thickness of about 1.24 mm). The increased-thickness cartridge can accommodate PCR reaction chambers of increased volume, including six-fold increases in volume as described above. The thicker cartridge implemented for viral load assay testing, however, can result in edge effect failures (outside sample lanes), reverse edge effect failures (inside sample lanes), and random failures. As described herein, when the compressible pad 314 was added to the top of the cartridge 300, the inventors of the present technology discovered that the results for viral load assay testing improved significantly. The compressible pad 314 as described herein can overcome long-term challenges with incorporating a pad onto the instrument or consumable (e.g., microfluidic cartridge). The compressible pad 314 can be considered a solution for pressure distribution effects associated with a microfluidic cartridges having increased thickness and increased-volume wells. While the example below describes a cartridge-based solution, in some embodiments, a compressible pad coupled to the optical detector 500 can include any of the features of the compressible pads described herein.

In this example, viral load assay testing included cartridge 200 as described herein, where the cartridge 200 has wider, deeper wells (e.g., PCR reaction chambers) than cartridge 100 described herein. The study design used a Geometry C Prototype cartridge, in which each reaction chamber has a width dimension of about 3.5 mm, a depth dimension of about 0.83 mm, a length dimension of about 10 mm, and a volume of about 25.2 μL. The study design included liquid master mix, and a cartridge hand-filled by a tester (as opposed to filled by an automated liquid dispenser). Each run tested both the first bank of reaction chambers and the second bank of reaction chambers of the cartridge. The test performed was PCR amplification. Testing was performed on 2 BD MAX™ instruments (Becton, Dickinson and Company, Franklin Lakes, N.J.). The viral load assay testing used two different RNA calibrator sequences, Hi Cal and Lo Cal.

FIGS. 7A-7C show results for viral load assay testing without a compressible pad according to the present technology. In this test, the cartridge 200 with wider, deeper wells was utilized without a compressible pad. FIG. 7A illustrates the quantification of the target nucleic acid in the sample. This illustrates the relationship between fluorescence and the number of amplification cycles on a logarithmic scale. The x-axis is the qCt score which illustrates the rate of change of the fluorescence. The number of cycles is indicated along the y-axis. During thermocycling such as for PCR, the quantity of the target nucleic acid doubles every cycle. Each color line on the graph indicates a separate reaction chamber. As described herein, a microfluidic cartridge can include 24 sample lanes arranged in 12 cartridge lanes, where each cartridge lane corresponds to a region of the cartridge 300 that includes 2 sample lanes 312. Each cartridge lane can include a reaction chamber in the first bank of reaction chamber and a reaction chamber in the second bank of reaction chambers. The qCt score of each reaction chamber in the same cartridge lane is assigned the same color in FIG. 7A. In each curve in FIG. 7A, there is a qCt score which indicates the first amplification cycle in which fluorescence is detected. In each curve in FIG. 7A, there is an y max EP score which indicates the maximum fluorescence unit in a final resting amplitude after a plurality of cycles. This data is also illustrated in FIGS. 7B and 7C.

FIG. 7B illustrates the individual qCt score for each reaction chamber of 24 reaction chambers of the cartridge 200. The upper graph indicates reaction chambers in the first bank of reaction chambers 226. The lower graph indicates reaction chambers in the second bank of reaction chambers 228. The twelve cartridge lanes are indicated on the y-axis. The qCt score which indicates the first amplification cycle in which fluorescence is detected is indicated on the x-axis. The qCt score for Hi Cal is fairly constant across the cartridge lanes, and the qCt score for Lo Cal is fairly constant across the cartridge lanes, although there is some wide variation in the first bank, in cartridge lanes 5 and 10. For the viral analyte sample, there is a significant variation in the qCt score, which indicates the first amplification cycle in which fluorescence is detected varies significantly among the 24 detection chambers. This variation in the reaction chambers is due to many factors, such as surface variations, poor contact between the cartridge and the heater substrate, poor compressibility of the cartridge by application of force, etc. In particular, reaction chambers in cartridge lanes 4-10 of the first bank have a higher qCt score for the viral analyte of interest than reaction chambers in cartridge lanes 1-3 and 11-12 of the first bank. In particular, reaction chambers in cartridge lanes 2, 3, 5, 10 of the second bank have a higher qCt score for the viral analyte of interest than reaction chambers in cartridge lanes 1, 4, 6-9, and 11-12 of the second bank.

FIG. 7C illustrates the y max EP for each reaction chamber. The upper graph indicates reaction chambers in the first bank of reaction chambers. The lower graph indicates reaction chambers in the second bank of reaction chambers. The twelve cartridge lanes are indicated on the y-axis. The y max EP score indicates the maximum fluorescence unit in a final resting amplitude after a plurality of cycles. The y max EP score for Hi Cal, the y max EP score for Lo Cal, and the y max EP score for the viral analyte sample have variation for the reaction chambers. The final resting amplitude is not consistent across the reaction chambers. This variation in the y max EP score for the reaction chambers is due to many factors, such as surface variations, poor contact between the cartridge and the heater substrate, poor compressibility of the cartridge by application of force, etc. This variation indicates inefficiencies with the PCR reaction, such that certain cartridge lanes did not meet the same maximum fluorescence. In particular, reaction chambers in cartridge lanes 1, 2, 11, 12 of the first bank have a higher y max EP scores for the viral analyte of interest than reaction chambers in cartridge lanes 3-10 of the first bank. In particular, reaction chambers in cartridge lanes 1, 4, 6, 7, 8, 9, 11, 12 of the second bank have a higher y max EP scores for the viral analyte of interest than reaction chambers in cartridge lanes 2, 3, 5, 10 of the second bank. This baseline illustrates the variations that can occur in microfluidic cartridge 200 when the compressible pad of the present technology is not implemented. Overall, amplification results in the reaction chambers are not consistent. For example, different reactions chambers are more efficient at PCR than other chambers. In FIGS. 7A-7B, the data is not clustered tightly which suggests wide variations in both the qCt score and the y max EP score.

FIGS. 8A-8D show results for viral load assay testing using a cartridge that implements a low durometer silicone compressible pad. The low durometer solid silicon used was a BISCO® HT-6210 silicone by Rogers Corporation. FIG. 8A illustrates an embodiment of the low durometer silicone compressible pad coupled to the top of the cartridge 200. FIG. 8B illustrates the quantification of the target nucleic acid in the sample. This illustrates the relationship between fluorescence and the number of amplification cycles on a logarithmic scale. The x-axis is the qCt score which illustrates the rate of change of the fluorescence. The y-axis is the number of cycles. In FIG. 8B, the data is clustered more tightly for initial amplification than FIG. 7A, suggesting less variation in the qCt score for each reaction chamber. In FIG. 8B, the data is not clustered tightly as the amplitudes become constant, which suggests wide variations in the y max EP score.

FIG. 8C illustrates the individual qCt score for each reaction chamber. The upper graph indicates reaction chambers in the first bank of reaction chambers 226. The lower graph indicates reaction chambers in the second bank of reaction chambers 228. The qCt score for Hi Cal and Lo Cal is fairly constant across the cartridge lanes. For the viral analyte sample, however, there is variation in the qCt score in cartridge lanes 5 and 7 of the first bank.

FIG. 8D illustrates the y max EP for each reaction chamber. The upper graph indicates reaction chambers in the first bank of reaction chambers. The lower graph indicates reaction chambers in the second bank of reaction chambers. The y max EP score for Lo Cal and the y max EP score for the viral analyte sample have variation for the reaction chambers in the first bank. The maximum fluorescence in a final resting amplitude after a plurality of cycles is not consistent. In particular, reaction chambers in cartridge lanes 1-3, 9-12 of the first bank have a higher y max EP scores for the viral analyte and Lo Cal than reaction chambers in cartridge lanes 4-8 of the first bank. Accordingly, the low durometer silicone compressible pad produces inconsistent PCR reactions as indicated in the graphs. In this example using the low durometer silicone compressible pad, different reactions chambers are more efficient at PCR than other chambers.

FIGS. 9A-9D show results for viral load assay testing using a cartridge that implements a compressible pad made of PORON® foam. FIG. 9A illustrates an embodiment of the PORON® foam compressible pad coupled to the top of the cartridge 200. PORON® foam is a fine pitch open cell urethane foam by Rogers Corporation. The material was PORON® Cellular Polyester Urethane 4790-92. FIG. 9B illustrates the quantification of the target nucleic acid in the sample. This illustrates the relationship between fluorescence and the number of amplification cycles on a logarithmic scale. The x-axis is the qCt score which illustrates the rate of change of the fluorescence. The y-axis is the number of cycles. In FIG. 9B, the data is clustered more tightly for initial amplification than FIGS. 7A and 8B, suggesting less variation in the qCt score for each reaction chamber. In FIG. 9B, the data is clustered more tightly for final amplification than FIGS. 7A and 8B, suggesting less variation in the y max EP score for each reaction chamber. In FIG. 9B, from left to right, the first cluster of lines relates to the Hi Cal, the second cluster of lines relates to the Lo Cal, and the third cluster of lines relates to the viral analyte sample.

FIG. 9C illustrates the individual qCt score for each reaction chamber. The upper graph indicates reaction chambers in the first bank of reaction chambers 226. The lower graph indicates reaction chambers in the second bank of reaction chambers 228. The qCt score for Hi Cal, Lo Cal, and the viral analyte sample is consistent across the cartridge lanes. For the Hi Cal RNA calibrator sequences, the initial amplitude, or in other words, the first detection, occurred at approximately the 20^{th} cycle. For the Lo Cal RNA calibrator sequences, the initial amplitude, or in other words, the first detection, occurred at approximately the 32^{nd} cycle. For the viral analyte sample sequences, the initial amplitude, or in other words, the first detection, occurred at approximately the 36^{th} cycle. These results are consistent for each cartridge lane. These results are consistent for each bank of the first bank and the second bank. These results are consistent for each reaction chamber of the 24 reaction chambers.

FIG. 9D illustrates the y max EP score for each reaction chamber. The upper graph indicates reaction chambers in the first bank of reaction chambers. The lower graph indicates reaction chambers in the second bank of reaction chambers. The y max EP score for Hi Cal, Lo Cal, and the viral analyte sample is consistent across the cartridge lanes (there are relatively small variances). For the Hi Cal RNA calibrator sequences, the maximum fluorescence unit in a final resting amplitude after a plurality of cycles is approximately 2000. For the Lo Cal RNA calibrator sequences, the maximum fluorescence unit in a final resting amplitude after a plurality of cycles is approximately 7000. For the viral analyte sample sequences, the maximum fluorescence unit in a final resting amplitude after a plurality of cycles is approximately 5500. These results are consistent for each cartridge lane. These results are consistent for each bank of the first bank and the second bank. These results are consistent for each reaction chamber of the 24 reaction chambers.

Additional testing was performed as outlined above, but using compressible pads of different materials. An additional test includes a compressible pad formed of graphite foil. The graphite foil was Tgon™ 820 by Laird. Another test included a compressible pad formed of fiberglass coated with thermally conductive silicon. The material was TF-1879 by ThermaCool®. A further test included a compressible pad formed of a silicone sponge. The material was BISCO® HT-800 silicone sponge by Rogers Corporation. Still another test included a compressible pad formed of thermal silicone sponge with a thermal coating. The material was R-10404 silicone sponge by ThermaCool®. The test design was similar to that outlined above for the low durometer silicone compressible pad (FIGS. 8A-8D) and the PORON® foam compressible pad (FIGS. 9A-9D).

In evaluating the materials for use with the compressible pad 314, unexpected results were achieved for a selected group of materials. In some embodiments, the compressible pad comprises a compressible material. In some embodiments, the suitable material for the compressible pad is selected based on Compression Force Deflection (in this case, the amount of stress (measured in psi) to deflect the material to 25% of its original height). The material can comprise a material that has a Compression Force Deflection less than 30 psi, less than 29 psi, less than 28 psi, less than 27 psi, less than 26 psi, less than 25 psi, less than 24 psi, less than 23 psi, less than 22 psi, less than 21 psi, 20 psi, less than 19 psi, less than 18 psi, less than 17 psi, less than 16 psi, less than 15 psi, less than 14 psi, less than 13 psi, less than 12 psi, less than 11 psi, less than 10 psi, less than 9 psi, less than 8 psi, less than 7 psi, less than 6 psi, less than 5 psi, less than 4 psi, less than 3 psi, less than 2 psi, less than 1 psi, etc. The material can comprise a material than has a Compression Force Deflection between 0 and 5 psi, between 5 and 10 psi, between 10 and 15 psi, between 15 and 20 psi, 20 and 25 psi, 25 and 30 psi, 0 and 3 psi, between 1 and 4 psi, between 2 and 5 psi, between 3 and 6 psi, 4 and 7 psi, between 5 and 8 psi, between 6 and 9 psi, between 7 and 10 psi, between 8 and 11 psi, between 9 and 12 psi, between 10 and 13 psi, between 11 and 14 psi, between 12 and 15 psi, between 13 and 16 psi, between 14 and 17 psi, between 15 and 18 psi, between 16 and 19 psi, between 17 and 20 psi, between 21 and 24 psi, between 22 and 25 psi, between 23 and 26 psi, between 24 and 27 psi, between 25 and 28 psi, between 26 and 29 psi, between 27 and 30 psi, etc. The material can comprise a material that has a Compression Force Deflection no greater than 5 psi, no greater than 10 psi, no greater than 15 psi, no greater than 20 psi, no greater than 25 psi, no greater than 30 psi, etc. In some embodiments, the test method is 0.51 cm/min (0.2″/min) Strain Rate with Force Measured @ 25% Deflection. In some embodiments, the range is between 0.3 and 3.5 psi (2-24 kPa). In some embodiments, the typical value is 1.7 psi (12 kPa).

As described herein, both a Durometer Shore A and Compression Force Deflection measured at 25% are measurements of compressibility. For these measurements, lower values indicate that the material is easier to compress, which was expected to lead to better pressure distribution and better PCR performance. Unexpectedly, the inventors discovered that Durometer Shore A was a poor indicator of suitable materials, see FIGS. 7B-7D. The low durometer silicone typically comprises a Hardness, Durometer, Shore “A” of 10 and the PORON® foam typically comprises a Hardness, Durometer, Shore “A” of less than 3. A person skilled in the art would expect that these materials would both easily compress and therefore lead to similar PCR performance. Unexpectedly, however, the performance for low durometer silicone was markedly different than the PORON® foam. Additional materials were tested and there was no correlation between Durometer Shore A and PCR performance.

Unexpectedly, Compression Force Deflection measured at 25% was an excellent indicator of suitable materials. The low durometer silicone typically comprises a Compression Force Deflection of about 30 psi and the PORON® foam typically comprises a Compression Force Deflection of between 2 and 5 psi. Additional materials were tested and there was a correlation between Compression Force Deflection and PCR performance. In particular, materials with a Compression Force Deflection of less than 30 psi measured at 25% deflection exhibited improved PCR performance. In some embodiments, materials with a Compression Force Deflection of between 0 and 20 psi had improved PCR performance.

As described herein, both Hardness, Durometer, Shore “A” and Compression Force Deflection measured at 25% determine characteristics of a material. These methods are useful for determining the relative hardness or firmness of a material. A person skilled in the art would expect that materials with a low Hardness, Durometer, Shore “A” and a low Compression Force Deflection measured at 25% would lead to better PCR performance. However, only Compression Force Deflection measured at 25% (not Hardness, Durometer, nor Shore “A”) provided a correlation to indicate suitable materials to improve PCR performance.

In some embodiments, the compressible pad comprises material properties as indicated below. In some embodiments, the compressible pad comprises a density according to ASTM D 3574-95, Test A. The density can range between 225 and 255 kg/m3. In some embodiments, the compressible pad comprises a thickness measured along the z-axis of the pad. The thickness can range from 0 to 5 mm, e.g., between 0 and 1 mm, between 1 and 2 mm, between 2 and 3 mm, between 3 and 4 mm, between 4 and 5 mm, approximately 3 mm (0.12″)+/−10%. In some embodiments, the compressible pad comprises Hardness, Durometer, Shore “O” according to ASTM D 2240-97 of 2. In some embodiments, the compressible pad comprises compression set, according to ASTM D 1667-90 Test D@ 23° C. (73° F.) of 2. In some embodiments, the compressible pad comprises compression set, according to ASTM D 3574-95 Test D@ 70° C. (158° F.) of 10. In some embodiments, the compressible pad comprises Resilience by Vertical Rebound, according to ASTM D 2632-96 of 4.

In some embodiments, the compressible pad comprises material properties as indicated below. In some embodiments, the compressible pad comprises tensile strength according to ASTM D412 of 120 psi (828 kPa). In some embodiments, the compressible pad comprises a thickness measured along the z-axis of the pad. The thickness can range from 0 to 5 mm, e.g., between 0 and 1 mm, between 1 and 2 mm, between 2 and 3 mm, between 3 and 4 mm, between 4 and 5 mm, approximately 1 mm (0.035″)+/−10%. In some embodiments, the compressible pad comprises elongation according to ASTM D412 of 150%. In some embodiments, the compressible pad comprises Hardness, Durometer, Shore “A” according to ASTM D 2240 of 13. In some embodiments, the compressible pad comprises compression deflection at 25% according to ASTM D1056 of 18 psi (125 kPa). In some embodiments, the compressible pad comprises compression set, according to ASTM D 1056 of 15. In some embodiments, the compressible pad comprises density according to ASTM 297 of 69 lbs/ft³ (1105 kg/m³).

In some embodiments, the compressible pad comprises material properties as indicated below. In some embodiments, the compressible pad comprises a thickness measured along the z-axis of the pad. The thickness can range from 0 to 5 mm, e.g., between 0 and 1 mm, between 1 and 2 mm, between 2 and 3 mm, between 3 and 4 mm, between 4 and 5 mm, approximately 1 mm (0.032″)+/−10%. In some embodiments, the compressible pad comprises elongation according to ASTM D412 of 80%. In some embodiments, the compressible pad comprises compression deflection at 25% according to ASTM D1056 of 9 psi (62 kPa). In some embodiments, the compressible pad comprises compression set, according to ASTM D 1056 of less than 1 at 70° C. and less than 5 at 100° C. In some embodiments, the compressible pad comprises density according to ASTM 1056 of 22 lbs/ft³ (352 kg/m³). In some embodiments, the compressible pad comprises material properties including a range of any two values herein. In some embodiments, the compressible pad comprises material properties including any value with +/−50% of the values herein.

Embodiments of the compressible pad of the present technology are designed to improve thermal transfer between a microfluidic cartridge and an associated heat source (for example, an array of heat sources underlying the microfluidic cartridge). As described herein, viral load is a numerical expression of the quantity of virus in a given volume. Microfluidic cartridges designed to determine the viral load, such as through PCR, may require wider, deeper wells for a larger reaction volume and to increase target detection. In these situations, the thermal transfer between the microfluidic cartridge with wider, deeper wells and the underlying heat source becomes critically important. Contact can be improved by a more even pressure distribution so that each PCR reaction chamber is in optimal contact with the underlying heat source. A uniform distribution of pressure can help to prevent poor repeatability of thermal cycling protocols between sample lanes, cartridge lanes, or cartridges. A uniform distribution of pressure can also avoid hot spots or heat transfer inefficiencies due to conductivity through air.

As described herein, the compressible pad of the present technology can be located on the top surface or bottom surface of the microfluidic cartridge. In some embodiments, the compressible pad on the bottom surface of the microfluidic cartridge can reduce thermal transfer. In some embodiments, the compressible pad on the top surface of the microfluidic cartridge can require windows or other cutouts to allow for optical detection. In some embodiments, the compressible pad is made as large as possible, for example co-extensive with the surface area of the label as described herein. In some embodiments, the compressible pad can comprise at least 50% of the surface area of a surface of the cartridge (e.g., 50% of the top surface of the cartridge), at least 60% of the surface, at least 70% of the surface, at least 80% of the surface, or at least 90% of the surface, etc.

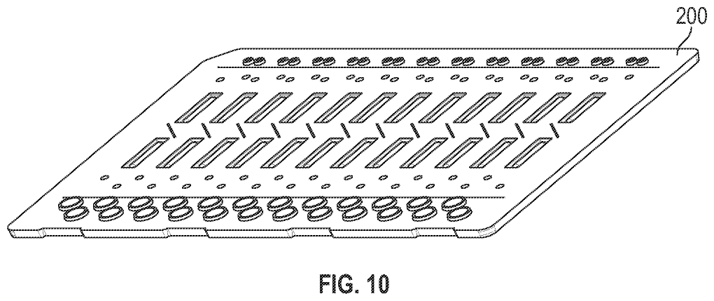

Another non-limiting implementation of a microfluidic cartridge according to the present technology will now be described with reference to FIGS. 10-37. FIGS. 10-37 show views of the microfluidic cartridge 200 containing twenty-four independent sample lanes.

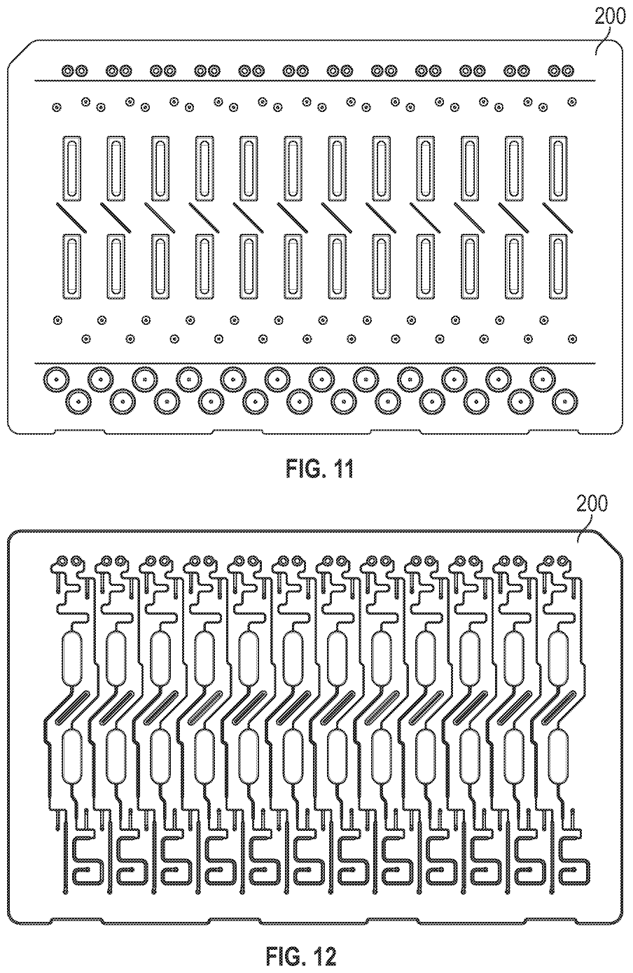

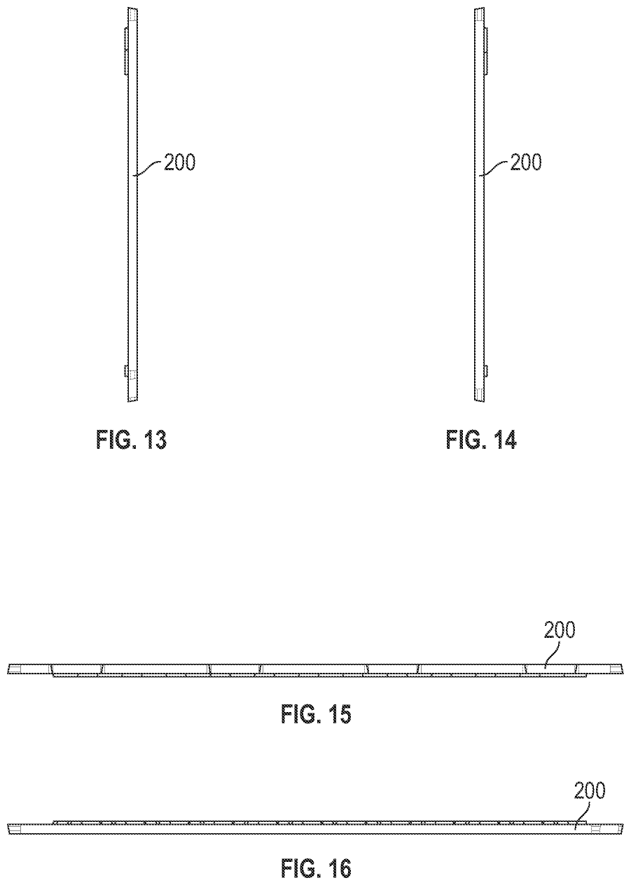

FIGS. 10-16 show a first embodiment of the microfluidic cartridge 200 without a compressible pad. FIG. 10 is a perspective view. FIG. 11 is a top view. FIG. 12 is a bottom view. FIG. 13 is a first side view. FIG. 14 is a second side view. FIG. 15 is a third side view. FIG. 16 is a fourth side view.

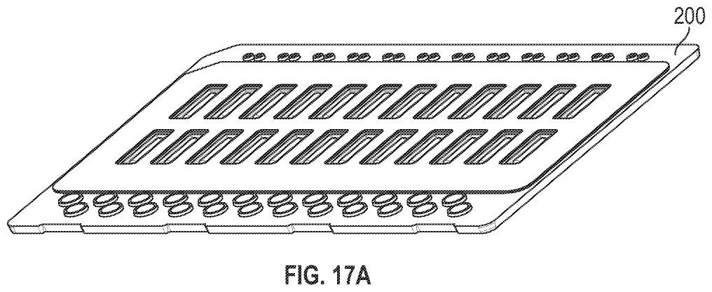

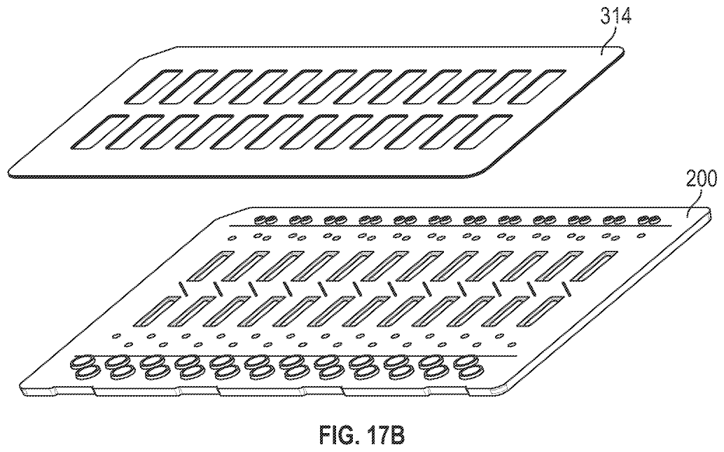

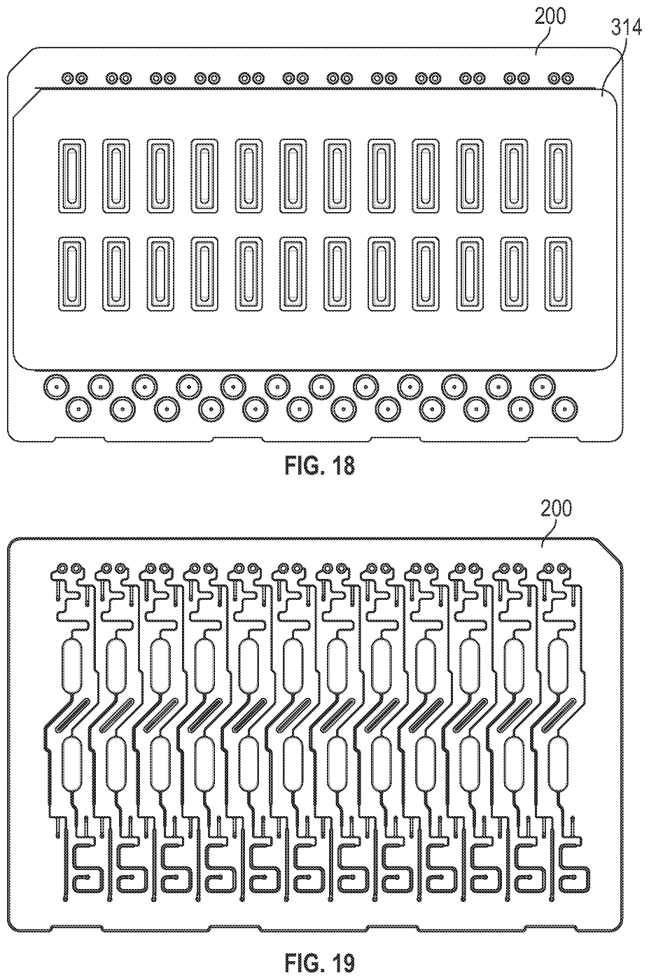

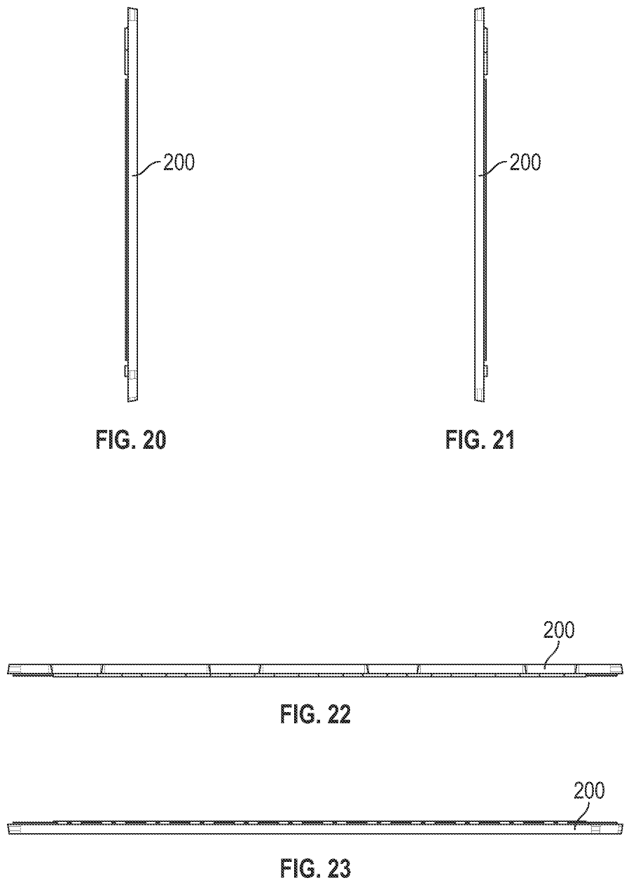

FIGS. 17-23 show a second embodiment of the microfluidic cartridge 200 with a compressible pad. FIG. 17A is a perspective view. FIG. 17B is an exploded view. FIG. 18 is a top view. FIG. 19 is a bottom view. FIG. 20 is a first side view. FIG. 21 is a second side view. FIG. 22 is a third side view. FIG. 23 is a fourth side view.

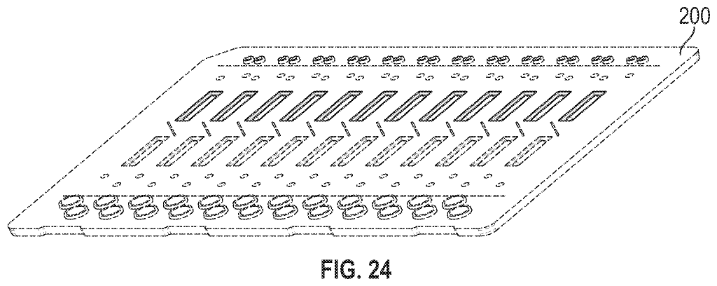

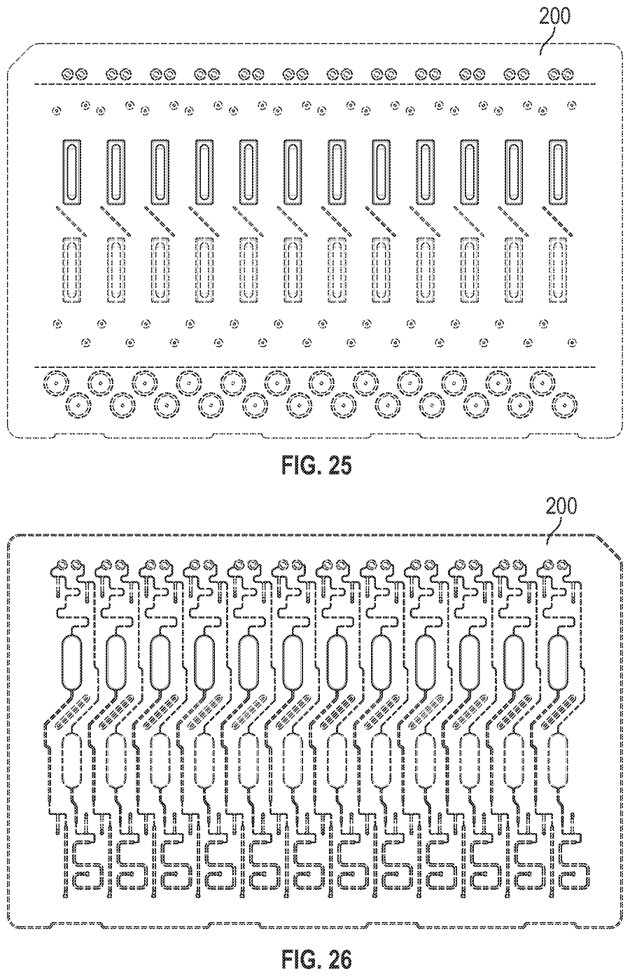

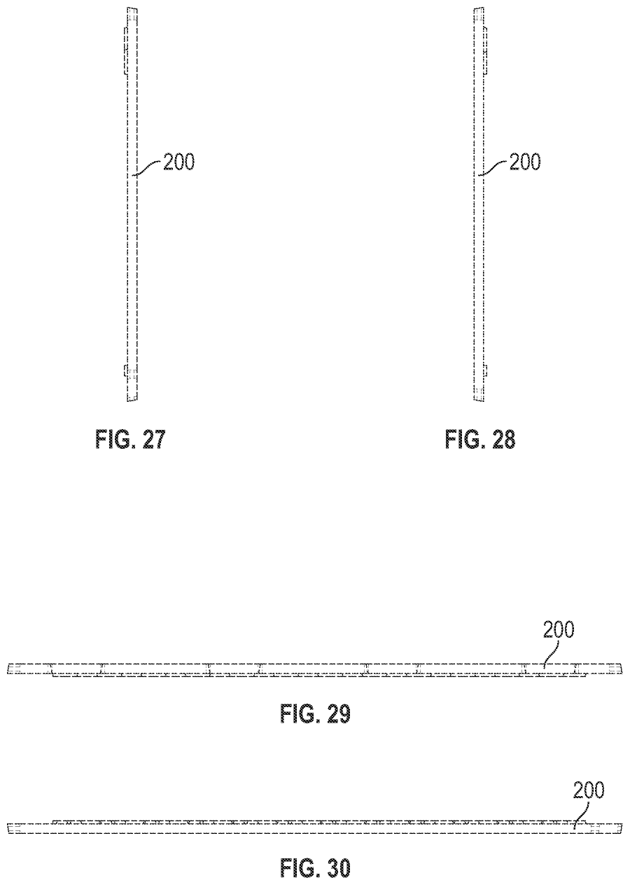

FIGS. 24-30 show additional views of the microfluidic cartridge of FIG. 10. FIG. 24 is a perspective view. FIG. 25 is a top view. FIG. 26 is a bottom view. FIG. 27 is a first side view. FIG. 28 is a second side view. FIG. 29 is a third side view. FIG. 30 is a fourth side view.

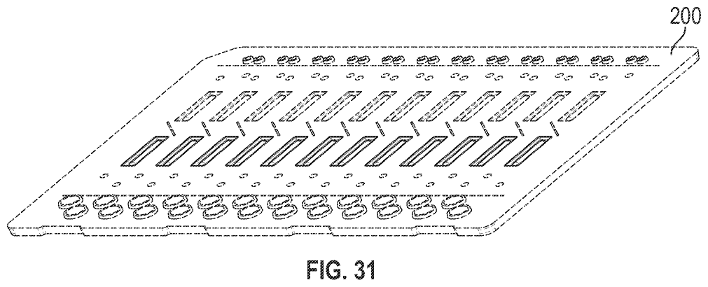

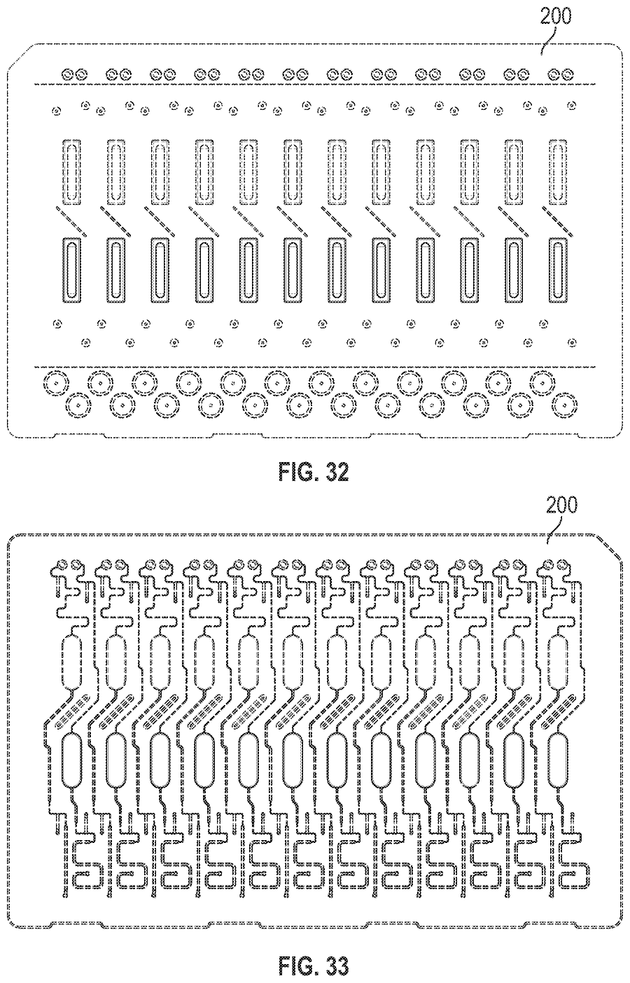

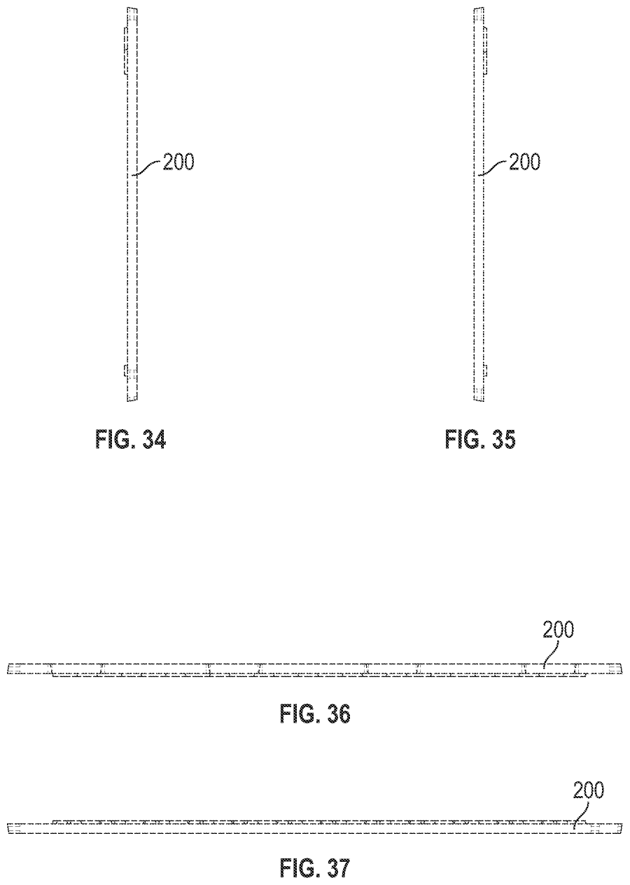

FIGS. 31-37 show additional views of the microfluidic cartridge of FIG. 10. FIG. 31 is a perspective view. FIG. 32 is a top view. FIG. 33 is a bottom view. FIG. 34 is a first side view. FIG. 35 is a second side view. FIG. 36 is a third side view. FIG. 37 is a fourth side view. Broken lines are used to illustrate features of the cartridge which form no part of the claimed design.

The present disclosure relates to molecular diagnostic test devices, systems, and methods to determine the presence and/or quantity of an analyte of interest in a sample. As used herein, “analyte” generally refers to a substance to be detected. For instance, analytes may include antigenic substances, haptens, antibodies, and combinations thereof. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles, and metabolites of or antibodies to any of the above substances.

Specific examples of analytes include, but are not limited to: Group B Streptococcal disease, Chlamydia trachomatis, Neisseria gonorrhoeae, Trichomonas vaginalis, Bacterial Vaginosis, Candida group, Candida Glabrata, Candida krusei, Salmonella spp., Shigella spp./enteroinvasive Escherichia coli (EIEC), Campylobacter spp. (jejuni and coli) and Shiga toxin producing organisms (STEC, Shigella dysenteriae), Yersinia enterocolitica, Enterotoxigenic E. coli (ETEC), Plesiomonas shigelloides, Vibrio (V. vulnuficus/V. parahaemolyticus/V. cholerae), Giardia lamblia, Cryptosporidium spp. (C. parvum and C. hominis), Entamoeba histolytica, Norovirus, Rotavirus, Adenovirus (40/41), Sapovirus and Human Astrovirus, Clostridium difficile toxin B gene (tcdB), MRSA, Staphylococcus aureus. Additional specific examples of analytes include, but are not limited to: ferritin; creatinine kinase MB (CK-MB); human chorionic gonadotropin (hCG); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein (CRP); lipocalins; IgE antibodies; cytokines; TNF-related apoptosis-inducing ligand (TRAIL); vitamin B2 micro-globulin; interferon gamma-induced protein 10 (IP-10); interferon-induced GTP-binding protein (also referred to as myxovirus (influenza virus) resistance 1, MX1, MxA, IFI-78K, IFI78, MX, MX dynamin like GTPase 1); procalcitonin (PCT); glycated hemoglobin (Gly Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyhene. Additional analytes may be included for purposes of biological or environmental substances of interest.

The foregoing description is intended to illustrate various aspects of the present technology. It is not intended that the examples presented herein limit the scope of the present technology. The technology now being fully described, it will be apparent to one of ordinary skill in the art that many changes and modifications can be made thereto without departing from the spirit or scope of the appended claims.

What is claimed is:

1. A microfluidic cartridge comprising a first side and an opposing, second side, comprising:
a first amplification chamber;
a second amplification chamber;
a first inlet disposed on the first side, in fluid communication with the first amplification chamber;
a second inlet disposed on the first side, in fluid communication with the second amplification chamber; and
a compressible pad disposed on the first side, the compressible pad configured to provide more thorough and consistent heat transfer to the first amplification chamber and the second amplification chamber from a plurality of contact heat sources in contact with the second side of the microfluidic cartridge, the compressible pad including a first window above the first amplification chamber and a second window above the second amplification chamber, the first window and the second window configured to allow light to be transmitted through the first side of the microfluidic cartridge to and from the first amplification chamber and the second amplification chamber, respectively.

2. The microfluidic cartridge of claim 1, wherein the first amplification chamber and the second amplification chamber have a volume of about 25 μL.

3. The microfluidic cartridge of claim 1, wherein the first amplification chamber and the second amplification chamber have a width dimension of about 3.5 mm, a depth dimension of about 0.83 mm, and a length dimension of about 10 mm.

4. The microfluidic cartridge of claim 1, wherein the microfluidic cartridge comprises a label above the compressible pad.

5. The microfluidic cartridge of claim 1, wherein the first amplification reaction chamber, the second amplification reaction chamber, the first inlet, and the second inlet are formed in a rigid substrate layer, and wherein the second side of the microfluidic cartridge comprises a flexible laminate layer below the first amplification chamber and the second amplification chamber.

6. The microfluidic cartridge of claim 1, wherein the compressible pad comprises a material with a Compression Force Deflection less than 30 psi.

7. The microfluidic cartridge of claim 1, wherein the compressible pad comprises a material with a Compression Force Deflection less than 20 psi.

8. The microfluidic cartridge of claim 1, wherein the compressible pad improves pressure distribution from a component of a diagnostic testing apparatus.

9. The microfluidic cartridge of claim 1, wherein application of pressure to the compressible pad is configured to increase uniformity of the application of heat from the plurality of contact heat sources to the first amplification chamber and the second amplification chamber.

10. The microfluidic cartridge of claim 1, wherein the compressible pad increases uniformity of the application of heat to the first amplification chamber and the second amplification chamber.

11. The microfluidic cartridge of claim 1, wherein the compressible pad enhances PCR amplification which relies on rapid temperature cycling.

12. A method for amplifying on a plurality of polynucleotide-containing samples, the method comprising:
introducing the plurality of samples into a microfluidic cartridge, wherein the cartridge comprises a plurality of amplification chambers configured to permit thermal cycling of the plurality of samples independently of one another;
moving the plurality of samples into the respective plurality of amplification chambers;
amplifying polynucleotides contained with the plurality of samples, by application of successive heating and cooling cycles to the amplification chambers; and
compressing a pad of the microfluidic cartridge during amplification.

13. The method of claim 12, further comprising applying pressure to the compressible pad to increase contact between the microfluidic cartridge and a substrate comprising one or more heaters.

14. The method of claim 12, further comprising applying pressure to the compressible pad to increase thermal uniformity.

15. The method of claim 12, further comprising applying pressure to the compressible pad to enhance amplification of the plurality of polynucleotide-containing samples.

16. A system comprising
a microfluidic cartridge, comprising:
a first PCR reaction chamber;
a second PCR reaction chamber;
a first inlet, in fluid communication with the first PCR reaction chamber;
a second inlet, in fluid communication with the second PCR reaction chamber; and
a compressible pad,
wherein the microfluidic cartridge is configured for use with an apparatus comprising:
a bay configured to receive the microfluidic cartridge;
at least one heat source thermally coupled to the cartridge and configured to apply heat cycles that carry out PCR on one or more polynucleotide-containing sample in the microfluidic cartridge;
a detector configured to detect presence of one or more polynucleotides in the one or more samples; and
a processor coupled to the heat source and configured to control heating of one or more regions of the microfluidic cartridge.

17. The system of claim 16, wherein the compressible pad is configured to improve contact between the bay and the microfluidic cartridge.

18. The system of claim 16, wherein the compressible pad is configured to improve contact between the at least one heat source and the microfluidic cartridge.

19. The system of claim 16, wherein the compressible pad is configured to be compressed by the detector which is disposed above the microfluidic cartridge during detection.

20. The system of claim 16, wherein the detector is configured to move down and make physical contact with the microfluidic cartridge to compress the compressible pad.

21. The system of claim 16, wherein the cartridge is configured to move up and make physical contact with the detector to compress the compressible pad.

22. The system of claim 16, wherein the compressible pad is configured to be compressed by another component of the apparatus which applies pressure to the microfluidic cartridge.
